(19) United States
(12) Patent Application Publication
(10) Pub. No.: US 2009/0176732 A1
(43) Pub. Date: Jul. 9, 2009

(54) PROTECTED NUCLEOTIDE ANALOGS

(75) Inventors: Leonid Beigelman, San Mateo, CA (US); Lawrence Blatt, San Francisco, CA (US); Harri Lonnberg, Turku (FI)

Correspondence Address: KNOBBE MARTENS OLSON & BEAR LLP, 2040 MAIN STREET, FOURTEENTH FLOOR, IRVINE, CA, 92614, US

(73) Assignee: Alios BioPharma Inc., South San Francisco, CA (US)

(21) Appl. No.: 12/340,408

(22) Filed: Dec. 19, 2008

Related U.S. Application Data
(60) Provisional application No. 61/016,352, filed on Dec. 21, 2007.

(51) Int. Cl. A61K 31/7056; C07H 19/10; C07H 19/056; A61K 31/7072; C07C 69/02

(52) U.S. Cl. 514/51; 536/26.8; 536/26.9; 514/52; 560/129

(57) ABSTRACT

Disclosed herein are nucleotide analogs with one or more protecting groups, methods of synthesizing nucleotide analogs with one or more protecting groups and methods of treating diseases and/or conditions such as viral infections, cancer, and/or parasitic diseases with the nucleotide analogs with one or more protecting groups.

This application claims priority to U.S. Provisional Application No. 61/016,352, entitled “PROTECTED NUCLEOTIDE ANALOGS,” filed on Dec. 21, 2007; which is incorporated herein by reference in its entirety, including any drawings.

### BACKGROUND

1. Field

The present application relates to the fields of chemistry, biochemistry and medicine. More particularly, disclosed herein are nucleotide analogs with one or more protecting groups, pharmaceutical compositions that include one or more nucleotide analogs with one or more protecting groups and methods of synthesizing the same. Also disclosed herein are methods of treating diseases and/or conditions with the nucleotide analogs with one or more protecting groups.

2. Description of the Related Art

Nucleoside analogs are a class of compounds that have been shown to exert antiviral and anticancer activity both in vitro and in vivo, and thus, have been the subject of widespread research for the treatment of viral infections and cancer. Nucleoside analogs are therapeutically inactive compounds that are converted by host or viral enzymes to their respective active anti-metabolites, which, in turn, inhibit polymerases involved in viral or cell proliferation. The activation occurs by a variety of mechanisms, such as the addition of one or more phosphate groups and, or in combination with, other metabolic processes.

Nucleoside analogs suffer from several problems that limit their use in treating viral infections and cancer. Nucleoside analogs depend upon intracellular phosphorylation to be biologically active. The absence or low activity of the necessary enzymes for phosphorylation can hamper the conversation of the nucleoside analog to its biologically active form. In addition, nucleoside analogs must be able to penetrate cell membranes and gain access to the intracellular space to be effective as therapeutics. Some nucleoside analogs traverse cell membranes by diffusional processes, which are governed by the charge and lipophilicity of the molecule. Others enter the cell by interaction with transporters for nucleosides present in the cell membrane. However, nucleoside analogs characteristically exhibit poor membrane permeability and are poorly soluble in water, thus, limiting their ability to penetrate cells. Furthermore, when administered to patients, studies have shown that nucleoside analogs are toxic to the liver, bone marrow and nervous system.

Use of nucleotide analogs overcomes the problem of the initial phosphorylation step. Nucleotide analogs are also structurally and metabolically closer to the therapeutically active form. However, the negatively charged phosphate on the nucleotide analogs severely limits the penetration of the nucleotide analogs into the cells. Prior attempts to neutralize the charge on the phosphate have resulted in nucleotide analogs with poor plasma stability, insufficient intracellular lability (releasability) and/or poor therapeutic efficacy.

### SUMMARY

An embodiment disclosed herein relates to a compound of Formula (I) or a pharmaceutically acceptable salt, prodrug or prodrug ester thereof:

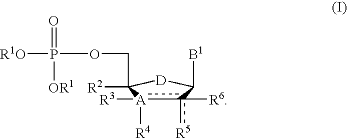

Another embodiment disclosed herein relates to a compound of Formula (II) or a pharmaceutically acceptable salt, prodrug or prodrug ester thereof:

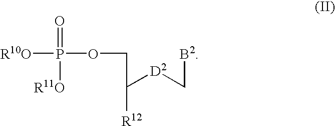

Still another embodiment disclosed herein relates to a compound of Formula (III) or a pharmaceutically acceptable salt, prodrug or prodrug ester:

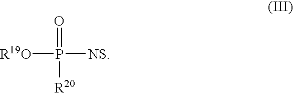

An embodiment disclosed herein relates to a compound selected from the following:

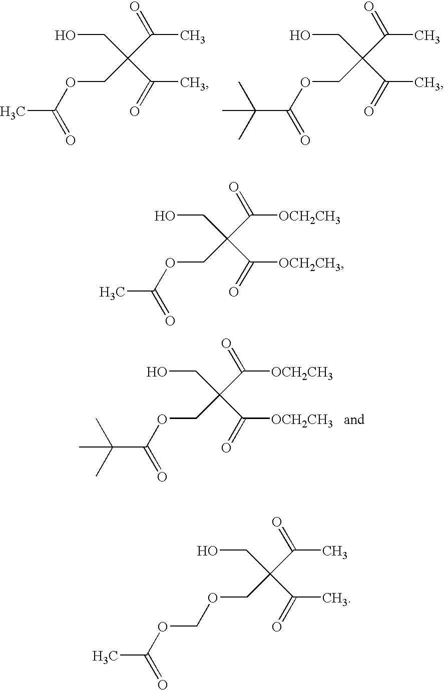

Another embodiment disclosed herein relates to a compound selected from the following:

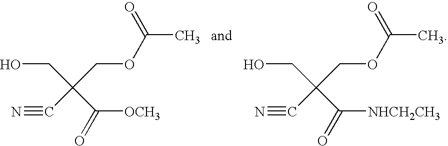

Some embodiments disclosed herein relate to methods of synthesizing a compound of Formula (I). Other embodiments disclosed herein relate to methods of synthesizing a compound of Formula (II).

An embodiments disclosed herein relates to a method that can include reacting a first reactant, wherein the first reactant can include a nucleoside with a phosphoamidite attached to the 5′-carbon or a protected nucleoside derivative with a phosphoamidite attached to the 5′-carbon or equivalent position, with a second reactant that can include a compound of Formula (G) having the structure

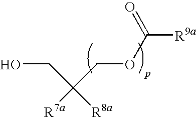

to form a compound having the structure

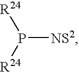

wherein at least one R²⁴ has the formula

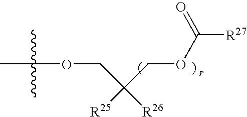

wherein R^{7a}, R^{8a}, R²⁵ and R²⁶ can be each independently —C≡N or an optionally substituted substituent selected from C₁₋₈ organylcarbonyl, C₁₋₈ alkoxycarbonyl and C₁₋₈ organylaminocarbonyl; R^{9a} and R²⁷ can be each independently hydrogen or an optionally substituted C₁₋₄-alkyl; p can be 1 or 2; r can be 1 or 2; NS² can be a nucleoside or a protected nucleoside derivative; and the other R²⁴ can be a biolabile group.

Another embodiments disclosed herein relates to a synthetic chemical method that can include, oxidizing the phosphorus in a compound having the structure

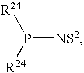

wherein at least one R²⁴ has the formula

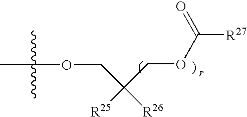

wherein R²⁵ and R²⁶ can be each independently —C≡N or an optionally substituted substituent selected from C₁₋₈ organylcarbonyl, C₁₋₈ alkoxycarbonyl and C₁₋₈ organylaminocarbonyl; R²⁷ can be hydrogen or an optionally substituted C₁₋₄-alkyl; r can be 1 or 2; NS² can be a nucleoside or a protected nucleoside derivative; and the other R²⁴ can be a biolabile group, to phosphorus (V).

Still another embodiment disclosed herein relates to a method that can include reacting a first reactant, wherein the first reactant can include a phosphite, with a second reactant that can include a compound of Formula (G) having the structure

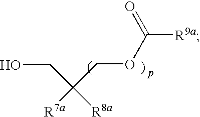

and a third reactant that can include a nucleoside or a protected nucleoside derivative; to form a compound having the structure

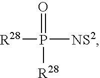

wherein at least one R²⁸ has the formula

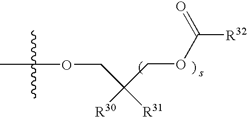

Wherein R^{7a}, R^{8a}, R³⁰ and R³¹ can be each independently —C≡N or an optionally substituted substituent selected from C₁₋₈ organylcarbonyl, C₁₋₈ alkoxycarbonyl and C₁₋₈ organylaminocarbonyl; R^{9a} and R³² can be each independently hydrogen or an optionally substituted C₁₋₄-alkyl; p can be 1 or 2; s can be 1 or 2; NS² can be a nucleoside or a protected nucleoside derivative; and the other R²⁸ is a biolabile group.

An embodiment disclosed herein relates to pharmaceutical compositions that can include one or more compounds of Formulae (I), (II), (III), (IV) and/or (V) or a pharmaceutically acceptable carrier, diluent, excipient or combination thereof.

Some embodiments disclosed herein relate to methods of ameliorating or treating a neoplastic disease that can include administering to a subject suffering from the neoplastic disease a therapeutically effective amount of one or more compound of Formulae (I), (II), (III), (IV) and/or (V) or a pharmaceutical composition that includes one or more compounds of Formulae (I), (II), (III), (IV) and/or (V).

Other embodiments disclosed herein relate to methods of inhibiting the growth of a tumor that can include administering to a subject having a tumor a therapeutically effective amount of one or more compound of Formulae (I), (II), (III), (IV) and/or (V) or a pharmaceutical composition that includes one or more compounds of Formulae (I), (II), (III), (IV) and/or (V).

Still other embodiments disclosed herein relate to methods of ameliorating or treating a viral infection that can include administering to a subject suffering from the viral infection a therapeutically effective amount of one or more compound of Formulae (I), (II), (III), (IV) and/or (V) or a pharmaceutical composition that includes one or more compounds of Formulae (I), (II), (III), (IV) and/or (V).

Yet still other embodiments disclosed herein relate to methods of ameliorating or treating a parasitic disease that can include administering to a subject suffering from the parasitic disease a therapeutically effective amount of one or more compound of Formulae (I), (II), (III), (IV) and/or (V) or a pharmaceutical composition that includes one or more compounds of Formulae (I), (II), (III), (IV) and/or (V).

### DETAILED DESCRIPTION

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, any “R” group(s) such as, without limitation, R¹, R^{1a} and R^{1b}, represent substituents that can be attached to the indicated atom. A non-limiting list of R groups include, but are not limited to, hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. An R group may be substituted or unsubstituted. If two “R” groups are covalently bonded to the same atom or to adjacent atoms, then they may be “taken together” as defined herein to form a cycloalkyl, cycloalkenyl, cycloalkynyl, aryl, heteroaryl or heteroalicyclyl group. For example, without limitation, if Rₐ and R_{b} of an NRₐR_{b} group are indicated to be “taken together”, it means that they are covalently bonded to one another at their terminal atoms to form a ring that includes the nitrogen:

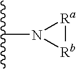

Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents.

The term “substituted” has its ordinary meaning, as found in numerous contemporary patents from the related art. See, for example, U.S. Pat. Nos. 6,509,331; 6,506,787; 6,500,825; 5,922,683; 5,886,210; 5,874,443; and 6,350,759; all of which are incorporated herein by reference for the limited purpose of disclosing suitable substituents that can be on a substituted group and standard definitions for the term “substituted.” Examples of suitable substituents include but are not limited to hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Each of these substituents can be further substituted. The other above-listed patents also provide standard definitions for the term “substituted” that are well-understood by those of skill in the art.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

As used herein, “alkenyl” refers to an alkyl group that contains in the straight or branched hydrocarbon chain one or more double bonds. An alkenyl group may be unsubstituted or substituted. When substituted, the substituent(s) may be selected from the same groups disclosed above with regard to alkyl group substitution unless otherwise indicated.

As used herein, “alkynyl” refers to an alkyl group that contains in the straight or branched hydrocarbon chain one or more triple bonds. An alkynyl group may be unsubstituted or substituted. When substituted, the substituent(s) may be selected from the same groups disclosed above with regard to alkyl group substitution unless otherwise indicated.

As used herein, “aryl” refers to a carbocyclic (all carbon) monocyclic or multicyclic aromatic ring system that has a fully delocalized pi-electron system. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof, unless the substituent groups are otherwise indicated.

As used herein, “heteroaryl” refers to a monocyclic or multicyclic aromatic ring system (a ring system with fully delocalized pi-electron system) that contain(s) one or more heteroatoms, that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

An “aralkyl” is an aryl group connected, as a substituent, via a lower alkylene group. The lower alkylene and aryl group of an aralkyl may be substituted or unsubstituted. Examples include but are not limited to benzyl, substituted benzyl, 2-phenylalkyl, 3-phenylalkyl, and naphtylalkyl.

A “heteroaralkyl” is heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, and imidazolylalkyl, and their substituted as well as benzo-fused analogs.

“Lower alkylene groups” are straight-chained tethering groups, forming bonds to connect molecular fragments via their terminal carbon atoms. Examples include but are not limited to methylene (—CH₂—), ethylene (—CH₂CH₂—), propylene (—CH₂CH₂CH₂—), and

As used herein, “cycloalkyl” refers to a completely saturated (no double or triple bonds) mono- or multi-cyclic hydrocarbon ring system. When composed of two or more rings, the rings may be joined together in a fused, bridged or spiro-connected fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, and the like. If substituted, the substituent(s) may be selected from those substituents indicated above with respect to substitution of an aryl group unless otherwise indicated.

As used herein, “cycloalkenyl” refers to a cycloalkyl group that contains one or more double bonds in the ring; although, if there is more than one, the double bonds cannot form a fully delocalized pi-electron system (otherwise the group would be “aryl,” as defined herein). When composed of two or more rings, the rings may be connected together in a fused, bridged or spiro-connected fashion. A cycloalkenyl group may be unsubstituted or substituted. When substituted, the substituent(s) may be selected from the substituents disclosed above with respect to an aryl group substitution unless otherwise indicated.

As used herein, “cycloalkynyl” refers to a cycloalkyl group that contains one or more triple bonds in the ring. If there is more than one triple bond, the triple bonds cannot form a fully delocalized pi-electron system. When composed of two or more rings, the rings may be joined together in a fused, bridged or spiro-connected fashion. A cycloalkynyl group may be unsubstituted or substituted. When substituted, the substituent(s) may be selected from the substituents disclosed above with respect to an aryl group substitution unless otherwise indicated.

As used herein, “heteroalicyclic” or “heteroalicyclyl” refers to a stable 3-to 18 membered monocyclic, bicyclic, tricyclic, or tetracyclic ring system which consists of carbon atoms and from one to five heteroatoms such as nitrogen, oxygen and sulfur. The “heteroalicyclic” or “heteroalicyclyl” may be joined together in a fused, bridged or spiro-connected fashion; and the nitrogen, carbon and sulfur atoms in the “heteroalicyclic” or “heteroalicyclyl” may be optionally oxidized; the nitrogen may be optionally quaternized; and the rings may also contain one or more double bonds provided that they do not form a fully delocalized pi-electron system throughout all the rings. Heteroalicyclyl or heteroalicyclic groups may be unsubstituted or substituted. When substituted, the substituent(s) may be one or more groups independently selected from: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, haloalkyl, haloalkoxy, trihalomethanesulfonyl, trihalomethanesulfonamido, and amino, including mono- and di-substituted amino groups, and the protected derivatives thereof. Examples of such “heteroalicyclic” or “heteroalicyclyl” groups include but are not limited to, azepinyl, acridinyl, carbazolyl, cinnolinyl, 1,3-dioxin, 1,3-dioxane, 1,4-dioxane, 1,2-dioxolanyl, 1,3-dioxolanyl, 1,4-dioxolanyl, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazolinyl, imidazolidine, isoxazoline, isoxazolidine, oxazoline, oxazolidine, oxazolidinone, thiazoline, thiazolidine, morpholinyl, oxiranyl, piperidinyl N-Oxide, piperidinyl, piperazinyl, pyrrolidinyl, pyrrolidone, pyrrolidione, 4-piperidonyl, pyrazoline, pyrazolidinyl, 2-oxopyrrolidinyl, tetrahydropyran, 4H-pyran, tetrahydrothiopyran, thiamorpholinyl, thiamorpholinyl sulfoxide, thiamorpholinyl sulfone, and their benzo-fused analogs (e.g., benzimidazolidinone, tetrahydroquinoline, 3,4-methylenedioxyphenyl).

A “(heteroalicyclyl)alkyl” is a heterocyclic or a heteroalicyclylic group connected, as a substituent, via a lower alkylene group. The lower alkylene and heterocyclic or a heterocyclyl of a (heteroalicyclyl)alkyl may be substituted or unsubstituted. Examples include but are not limited tetrahydro-2H-pyran-4-yl)methyl, (piperidin-4-yl)ethyl, (piperidin-4-yl)propyl, (tetrahydro-2H-thiopyran-4-yl)methyl, and (1,3-thiazinan-4-yl)methyl.

As used herein, “alkoxy” refers to the formula —OR wherein R is an alkyl is defined as above, e.g. methoxy, ethoxy, n-propoxy, 1-methylethoxy (isopropoxy), n-butoxy, iso-butoxy, sec-butoxy, tert-butoxy, and the like. An alkoxy may be substituted or unsubstituted.

As used herein, “acyl” refers to a hydrogen, alkyl, alkenyl, alkynyl, or aryl connected, as substituents, via a carbonyl group. Examples include formyl, acetyl, propanoyl, benzoyl, and acryl. An acyl may be substituted or unsubstituted.

As used herein, “hydroxyalkyl” refers to an alkyl group in which one or more of the hydrogen atoms are replaced by hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

As used herein, “haloalkyl” refers to an alkyl group in which one or more of the hydrogen atoms are replaced by halogen (e.g., mono-haloalkyl, di-haloalkyl and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

As used herein, “aryloxy” and “arylthio” refers to RO— and RS—, in which R is an aryl, such as but not limited to phenyl. Both an aryloxy and arylthio may be substituted or unsubstituted.

A “sulfenyl” group refers to an “—SR” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂-” group wherein X is a halogen.

A “trihalomethanesulfonamido” group refers to an “X₃CS(O)₂ RAN-” group wherein X is a halogen and R_{A} defined with respect to O-carboxy.

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “mercapto” group refers to an “—SH” group.

A “carbonyl” group refers to a C═O group.

An “S-sulfonamido” group refers to a “—SO₂NR_{A}R_{B}” group in which R_{A} and R_{B} can be the same as R defined with respect to O-carboxy. An S-sulfonamido may be substituted or unsubstituted.

An “N-sulfonamido” group refers to a “RSO₂N(R_{A})—” group in which R and R_{A} can be the same as R defined with respect to O-carboxy. A N-sulfonamido may be substituted or unsubstituted.

An “O-carbamyl” group refers to a “—OC(═O)NR_{A}R_{B}” group in which R_{A} and R_{B} can be the same as R defined with respect to O-carboxy. An O-carbamyl may be substituted or unsubstituted.

An “N-carbamyl” group refers to an “ROC(═O)NR_{A}—” group in which R and R_{A} can be the same as R defined with respect to O-carboxy. An N-carbamyl may be substituted or unsubstituted.

An “O-thiocarbamyl” group refers to a “—OC(═S)—NR_{A}R_{B}” group in which R_{A} and R_{B} can be the same as R defined with respect to O-carboxy. An O-thiocarbamyl may be substituted or unsubstituted.

An “N-thiocarbamyl” group refers to an “ROC(═S)NR_{A}—” group in which R and R_{A} can be the same as R defined with respect to O-carboxy. An N-thiocarbamyl may be substituted or unsubstituted.

A “C-amido” group refers to a “—C(═O)NR_{A}R_{B}” group in which R_{A} and R_{B} can be the same as R defined with respect to O-carboxy. A C-amido may be substituted or unsubstituted.

An “N-amido” group refers to a “RC(═O)NR_{A}—” group in which R and R_{A} can be the same as R defined with respect to O-carboxy. An N-amido may be substituted or unsubstituted.

As used herein, “organylcarbonyl” refers to a group of the formula —C(═O)Rₐ wherein Rₐ can be alkyl, alkenyl, alkynyl, cycloalkyl, cycloalkenyl, cycloalkynyl, aryl, heteroaryl, heteroalicyclyl, aralkyl, or (heteroalicyclyl)alkyl. An organylcarbonyl can be substituted or unsubstituted.

The term “alkoxycarbonyl” as used herein refers to a group of the formula —C(═O)ORₐ wherein Rₐ can be the same as defined with respect to organylcarbonyl. An alkoxycarbonyl can be substituted or unsubstituted.

As used herein, “organylaminocarbonyl” refers to a group of the formula —C(═O)NHRₐ wherein Rₐ can be the same as defined with respect to organylcarbonyl. An organylaminocarbonyl can be substituted or unsubstituted.

As used herein, the term “levulinoyl” refers to a —C(═O)CH₂CH₂C(═O)CH₃ group.

The term “biolabile” group as used herein refers to a group or moiety that can be removed from the phosphorus atom on a nucleotide, such as a monophosphate nucleotide, after the nucleotide has penetrated a cell. For example, the biolabile group can be removed by esterases after entry into a intracellular space of cell. Suitable biolabile groups are known to those skilled in the art. In some embodiments, the biolabile group reduces the charge of the phosphate group. In an embodiment, the biolabile can form a phosphoester bond with the phosphate on the nucleotide. In some embodiments, the biolabile group can be removed via hydrolysis. Examples of biolabile groups include cholesterol, galactose, galactosamine, glucose, glucosamine, vitamins (e.g., folate), optionally substituted benzyl alcohols (such as alpha-ester-benzyl alcohols, alpha-acyl-benzyl alcohols, ring-substituted benzyl alcohols and combinations thereof) and optionally substituted phenols.

The term “halogen atom,” as used herein, means any one of the radio-stable atoms of column 7 of the Periodic Table of the Elements, i.e., fluorine, chlorine, bromine, or iodine, with bromine and chlorine being preferred.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

As used herein, the term “nucleoside” refers to a compound composed of any pentose or modified pentose moiety attached to a specific portion of a heterocyclic base, tautomer, or derivative thereof such as the 9-position of a purine, 1-position of a pyrimidine, or an equivalent position of a heterocyclic base derivative. Examples include, but are not limited to, a ribonucleoside comprising a ribose moiety and a deoxyribonucleoside comprising a deoxyribose moiety. In some instances, the nucleoside can be a nucleoside drug analog.

As used herein, the term “nucleoside drug analog” refers to a compound composed of a nucleoside that has therapeutic activity, such as antiviral, anti-neoplastic, anti-parasitic and/or antibacterial activity.

As used herein, the term “nucleotide” refers to a nucleoside having a phosphate ester substituted on the 5′-position or an equivalent position of a nucleoside derivative.

As used herein, the terms “protected nucleoside” and “protected nucleoside derivative” refers to a nucleoside and nucleoside derivative, respectively, in which one or more hydroxy groups attached to the ribose or deoxyribose ring are protected with one or more protecting groups. An example of protected nucleoside is an adenosine in which the oxygen at the 3′-position is protected with a protecting group such as methyl group or a levulinoyl group.

As used herein, the term “heterocyclic base” refers to a purine, a pyrimidine and derivatives thereof. The term “purine” refers to a substituted purine, its tautomers and analogs thereof. Similarly, the term “pyrimidine” refers to a substituted pyrimidine, its tautomers and analogs thereof. Exemplary purines include, but are not limited to, purine, adenine, guanine, hypoxanthine, xanthine, theobromine, caffeine, uric acid and isoguanine. Examples of pyrimidines include, but are not limited to, cytosine, thymine, uracil, and derivatives thereof. An example of an analog of a purine is 1,2,4-triazole-3-carboxamide.

Other non-limiting examples of heterocyclic bases include diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-fluorouracil, 5-bromouracil, pseudoisocytosine, isocytosine, isoguanine, and other heterocyclic bases described in U.S. Pat. Nos. 5,432,272 and 7,125,855, which are incorporated herein by reference for the limited purpose of disclosing additional heterocyclic bases.

As used herein, the term “protected heterocyclic base” refers to a heterocyclic base in which one or more amino groups attached to the base are protected with one or more suitable protecting groups and/or one or more —NH groups present in a ring of the heterocyclic base are protected with one or more suitable protecting groups. When more than one protecting group is present, the protecting groups can be the same or different.

The terms “derivative,” “variant,” or other similar terms refer to a compound that is an analog of the other compound.

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls (e.g., t-butoxycarbonyl (BOC)); arylalkylcarbonyls (e.g., benzyloxycarbonyl, benzoyl); substituted methyl ether (e.g. methoxymethyl ether); substituted ethyl ether; a substituted benzyl ether; tetrahydropyranyl ether; silyl ethers (e.g., trimethylsilyl, triethylsilyl, triisopropylsilyl, t-butyldimethylsilyl, or t-butyldiphenylsilyl); esters (e.g. benzoate ester); carbonates (e.g. methoxymethylcarbonate); sulfonates (e.g. tosylate, mesylate); acyclic ketal (e.g. dimethyl acetal); cyclic ketals (e.g., 1,3-dioxane or 1,3-dioxolanes); acyclic acetal; cyclic acetal; acyclic hemiacetal; cyclic hemiacetal; and cyclic dithioketals (e.g., 1,3-dithiane or 1,3-dithiolane).

“Leaving group” as used herein refers to any atom or moiety that is capable of being displaced by another atom or moiety in a chemical reaction. More specifically, in some embodiments, “leaving group” refers to the atom or moiety that is displaced in a nucleophilic substitution reaction. In some embodiments, “leaving groups” are any atoms or moieties that are conjugate bases of strong acids. Examples of suitable leaving groups include, but are not limited to, tosylates and halogens. Non-limiting characteristics and examples of leaving groups can be found, for example in Organic Chemistry, 2d ed., Francis Carey (1992), pages 328-331; Introduction to Organic Chemistry, 2d ed., Andrew Streitwieser and Clayton Heathcock (1981), pages 169-171; and Organic Chemistry, 5^{th} ed., John McMurry (2000), pages 398 and 408; all of which are incorporated herein by reference for the limited purpose of disclosing characteristics and examples of leaving groups.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference for the limited purpose describing procedures and preparation of suitable prodrug derivatives.

The term “pro-drug ester” refers to derivatives of the compounds disclosed herein formed by the addition of any of several ester-forming groups that are hydrolyzed under physiological conditions. Examples of pro-drug ester groups include pivaloyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference for the limited purpose of disclosing ester-forming groups that can form prodrug esters.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine, lysine, and the like.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound described herein having one or more double bond(s) generating geometrical isomers that can be defined as E or Z, each double bond may independently be E or Z a mixture thereof. Likewise, all tautomeric forms are also intended to be included.

An embodiment disclosed herein relates to a compound of Formula (I) or a pharmaceutically acceptable salt or prodrug thereof:

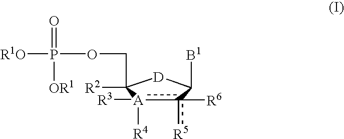

wherein: [symbol] can be a double or single bond; A can be selected from C (carbon), O (oxygen) and S (sulfur); B¹ can be an optionally substituted heterocyclic base or a derivative thereof; D can be C═CH₂ or O (oxygen); each R¹ can be each independently absent, hydrogen or

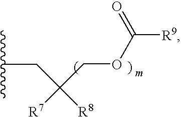

provided that at least one R¹ is

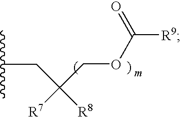

R² can be selected from hydrogen, azido, —CN, an optionally substituted C₁₋₄ alkyl and an optionally substituted C₁₋₄ alkoxy; R³ can be absent or selected from hydrogen, halogen, hydroxy and an optionally substituted C₁₋₄ alkyl; R⁴ can be absent or selected from hydrogen, halogen, azido, amino and hydroxy; R⁵ can be selected from hydrogen, halogen, hydroxy, —CN, —NC, an optionally substituted C₁₋₄ alkyl and an optionally substituted C₁₋₄ alkoxy; R⁶ can be absent or selected from hydrogen, halogen, hydroxy, —CN, —NC, an optionally substituted C₁₋₄ alkyl, an optionally substituted haloalkyl and an optionally substituted hydroxyalkyl, or when the bond to R⁵ indicated by [symbol] is a double bond, then R⁵ is a C₁₋₄ alkenyl and R⁶ absent; R⁷ and R⁸ can be each independently —C≡N or an optionally substituted substituent selected from C₁₋₈ organylcarbonyl, C₁₋₈ alkoxycarbonyl and C₁₋₈ organylaminocarbonyl; R⁹ can be hydrogen or an optionally substituted C₁₋₄-alkyl; and each m can be independently 1 or 2. In an embodiment, each m can be 1. In another embodiment, each m can be 2. In some embodiments, at least one m can be 1. In an embodiment, at least one m can be 2. In an embodiment, one m can be 1, and the other m, if present, can be 2.

In some embodiments, the optionally substituted C₁₋₄ alkyl can be selected from methyl, ethyl, n-propyl, isopropyl, n-butyl, iso-butyl and tert-butyl. In some embodiments, the optionally substituted C₁₋₄ alkoxy can be selected from methoxy, ethoxy, n-propoxy, isopropoxy, n-butoxy, iso-butoxy and tert-butoxy.

The substitutents on

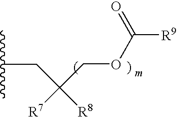

can vary. In some embodiments, R⁷ can be —C≡N and R⁸ can be an optionally substituted C₁₋₈ alkoxycarbonyl such as —C(═O)OCH₃. In other embodiments, R⁷ can be —C≡N and R⁸ can be an optionally substituted C₁₋₈ organylaminocarbonyl, for example, —C(═O)NHCH₂CH₃ and C(═O)NHCH₂CH₂phenyl. In still other embodiments, both R⁷ and R⁸ can be an optionally substituted C₁₋₈ organylcarbonyl. In an embodiment, both R⁷ and R⁸ can be —C(═O)CH₃. In yet still other embodiments, both R⁷ and R⁸ can be an optionally substituted C₁₋₈ alkoxycarbonyl. In an embodiment, both R⁷ and R⁸ can be —C(═O)OCH₂CH₃. In some embodiments, R⁹ can be an optionally substituted C₁₋₄-alkyl. In an embodiment, R⁹ can be methyl or tert-butyl.

Examples of suitable

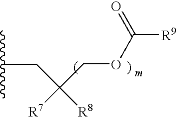

groups, include but are not limited to, the following:

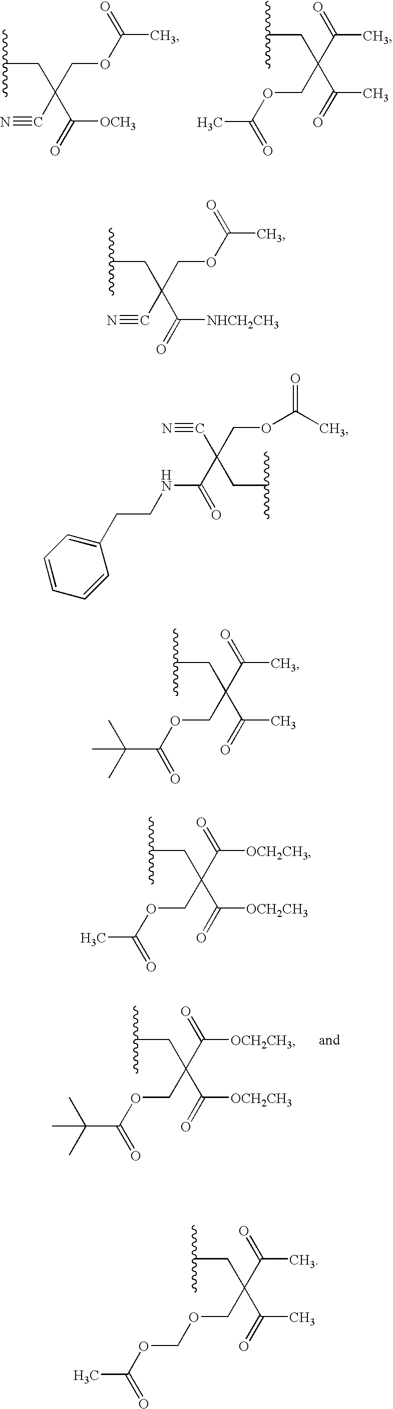

In an embodiment, at least one R¹ can be

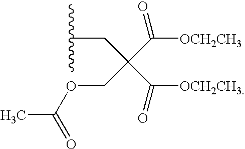

In another embodiment, at least one R¹ can be

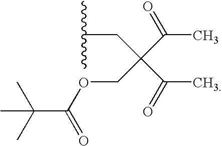

In still another embodiment, at least one R¹ can be

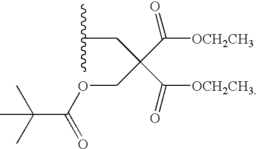

In yet still another embodiment, at least one R¹ can be

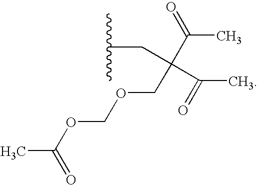

In an embodiment, at least one R¹ can be

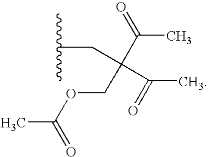

In some embodiments, both R¹ groups may be the same chemical moiety. In other embodiments, the R¹ groups may be different from one another. In an embodiment, both R¹ groups are

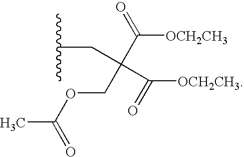

In another embodiment, both R¹ groups are

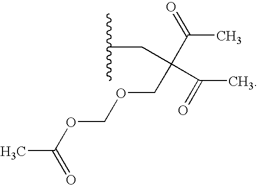

The substituent B¹ can also vary. In some embodiments, B¹ can be selected from:

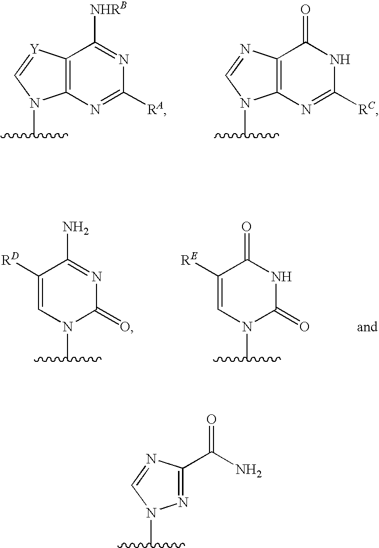

wherein: R^{A} can be hydrogen or halogen; R^{B} can be hydrogen, an optionally substituted C₁₋₄alkyl, or an optionally substituted C₃₋₈ cycloalkyl; R^{C} can be hydrogen or amino; R^{D} can be hydrogen or halogen; R^{E} can be hydrogen or an optionally substituted C₁₋₄alkyl; and Y can be N (nitrogen) or CR^{F}, wherein R^{F} hydrogen, halogen or an optionally substituted C₁₋₄ alkyl.
In an embodiment, B¹ can be

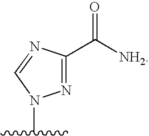

In another embodiment, B¹ can be

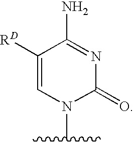

In yet another embodiment, B¹ can be

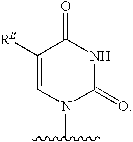

In some embodiments, the compound of Formula (I) can be an anti-neoplastic agent. In other embodiments, the compound of Formula (I) can be an anti-viral agent. In still other embodiments, the compound of Formula (I) can be an anti-parasitic agent.

Exemplary of compounds of Formula (I) are shown below. The compounds shown below are examples and do not represent all compounds of Formula (I).

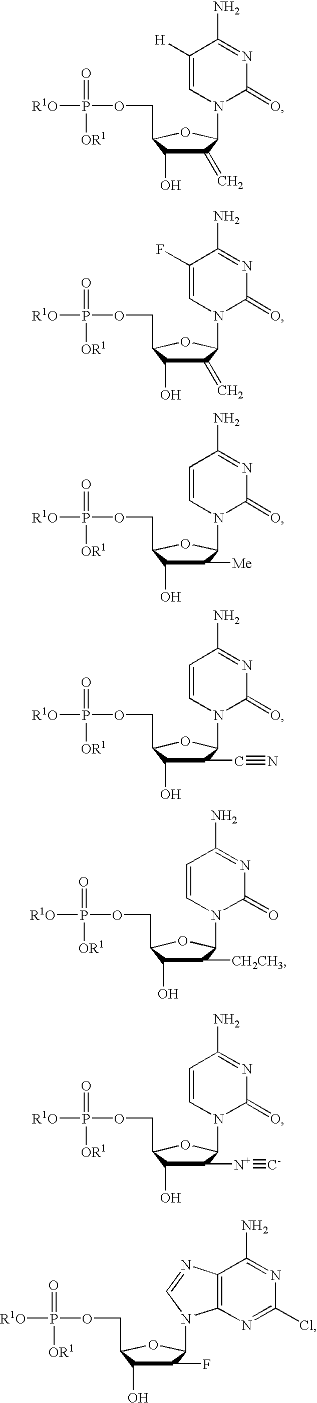

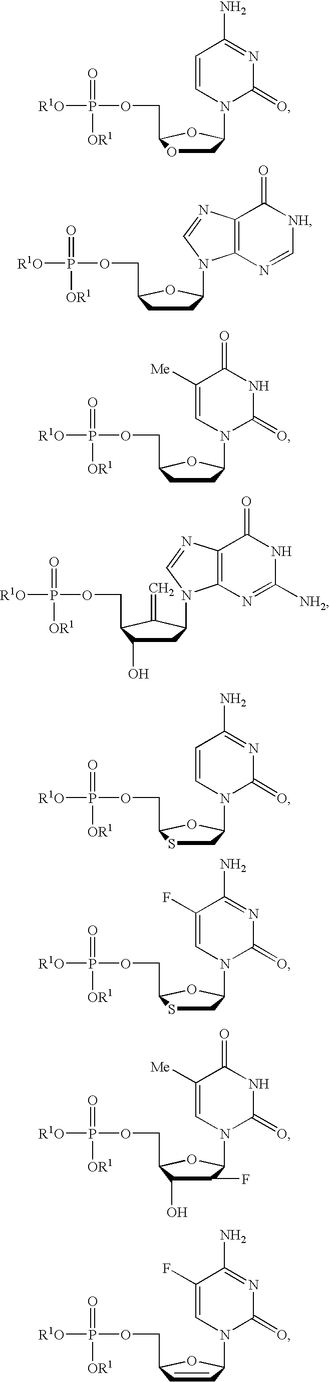

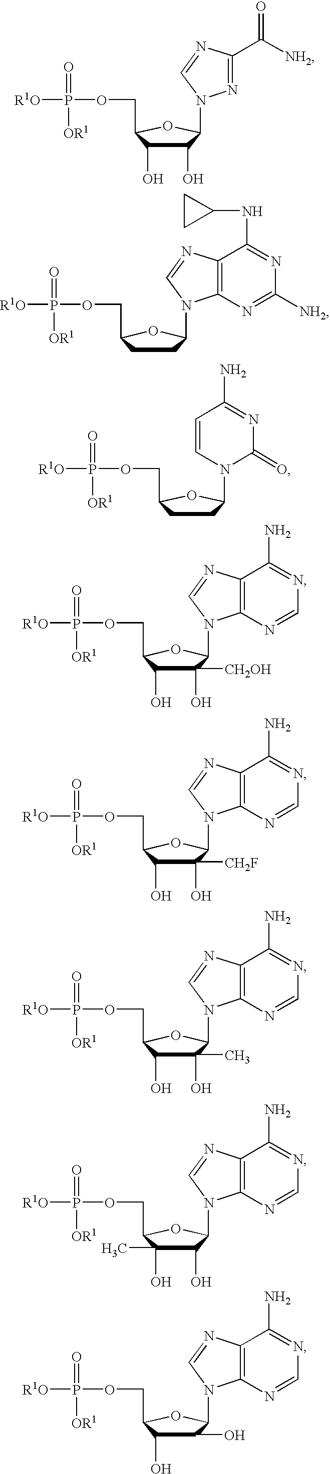

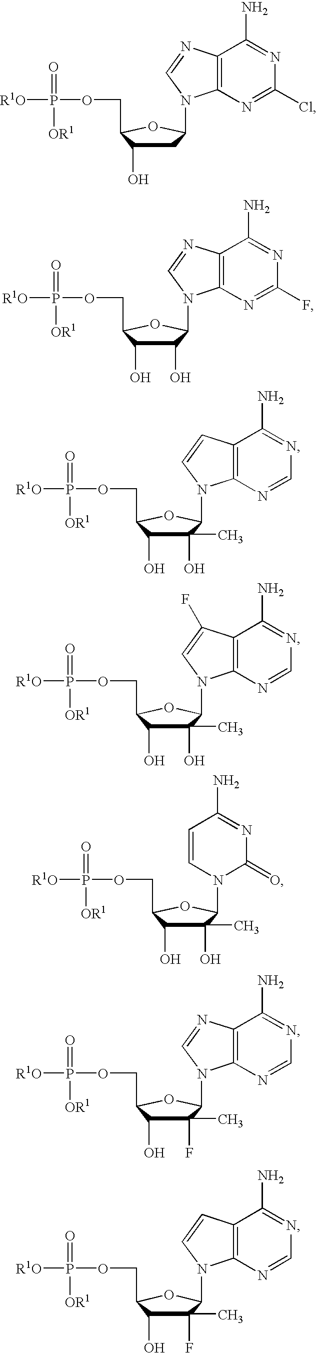

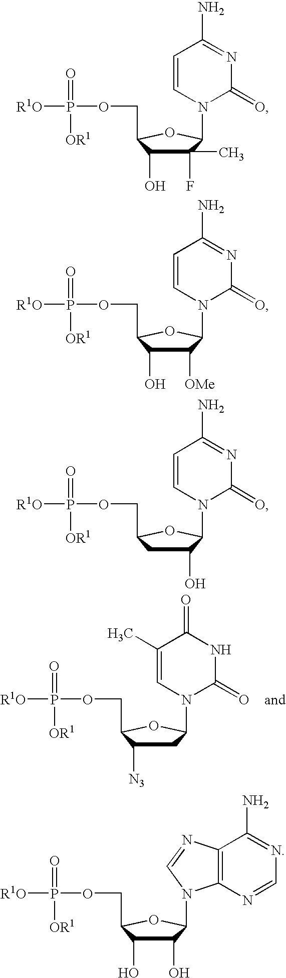

In an embodiment, a compound of Formula (I) can be one of the following compounds:

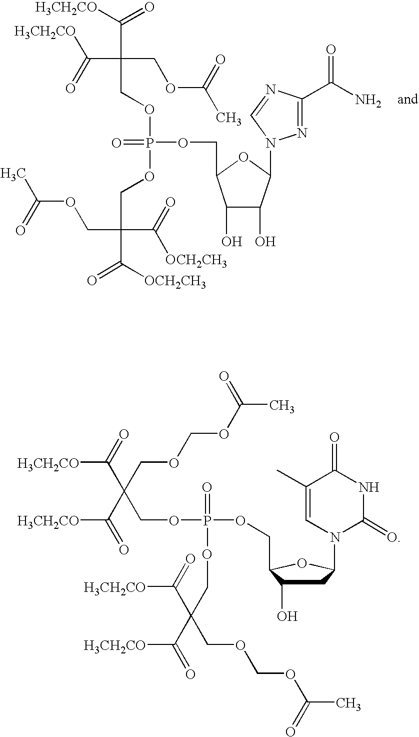

An embodiment disclosed herein relates to a compound of Formula (II) or a pharmaceutically acceptable salt or prodrug thereof:

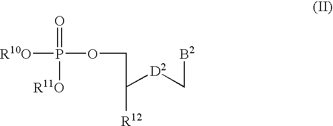

wherein: B² can be an optionally substituted heterocyclic base or a derivative thereof; D² can be 0 (oxygen) or —CH₂; R¹⁰ and R¹¹ are each independently absent, hydrogen or

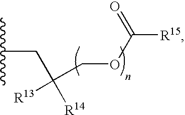

provided that at least one of R¹⁰ and R¹¹ are

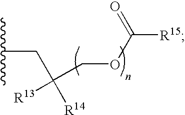

R¹² can be hydrogen or —(CH₂)—OH; R¹³ and R¹⁴ can be each independently —C≡N or an optionally substituted substituent selected from C₁₋₈ organylcarbonyl, C₁₋₈ alkoxycarbonyl and C₁₋₈ organylaminocarbonyl; R¹⁵ can be hydrogen or an optionally substituted C₁₋₄-alkyl; and each n can be independently 1 or 2. In an embodiment, each n can be 1. In another embodiment, each n can be 2. In some embodiments, at least one n can be 1. In an embodiment, at least one n can be 2. In some embodiments, one n can be 1, and the other n, if present, can be 2.

Examples of suitable

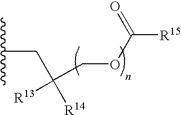

groups, include but are not limited to, the following:

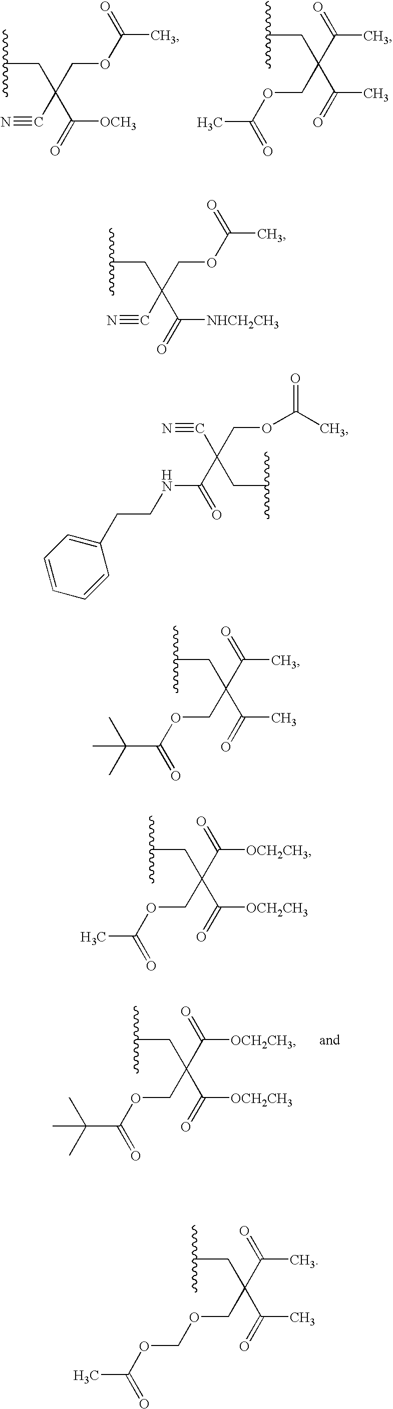

In an embodiment, R¹⁰ and/or R¹¹ can be

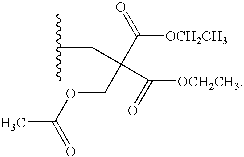

In another embodiment, R¹⁰ and/or R¹¹ can be

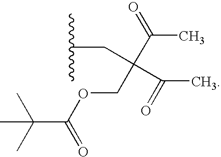

In still another embodiment, R¹⁰ and/or R¹¹ can be

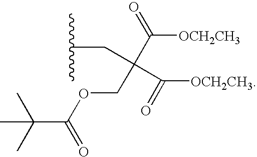

In yet still another embodiment, R¹⁰ and/or R¹¹ can be

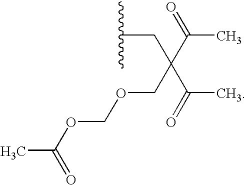

In an embodiment, R¹⁰ and/or R¹¹ can be

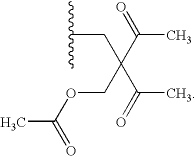

In some embodiments, both R¹⁰ and R¹¹ groups may be the same chemical moiety. In other embodiments, the R¹⁰ and R¹¹ groups may be different from one another. In an embodiment, both R¹⁰ and R¹¹ groups are

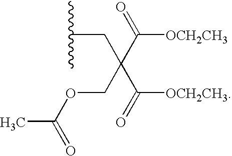

In another embodiment, both R¹⁰ and R¹¹ groups are

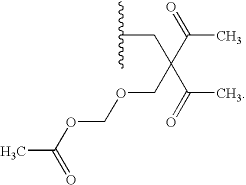

In some embodiments, R¹³ can be —C≡N and R¹⁴ can be an optionally substituted C₁₋₈ alkoxycarbonyl such as —C(═O)OCH₃. In other embodiments, R¹³ can be —C≡N and R¹⁴ can be an optionally substituted C₁₋₈ organylaminocarbonyl, for example, —C(═O)NHCH₂CH₃ and —C(═O)NHCH₂CH₂phenyl. In still other embodiments, both R¹³ and R¹⁴ can be a C₁₋₈ organylcarbonyl. In an embodiment, the C₁₋₈ organylcarbonyl can be —C(═O)CH₃. In yet still other embodiments, both R¹³ and R¹⁴ can be an optionally substituted alkoxycarbonyl such as —C(═O)OCH₂CH₃. In some embodiments, R¹⁵ can be an optionally substituted C₁₋₄ alkyl. In an embodiment, R¹⁵ can be methyl or tert-butyl.

In some embodiments, B² can be selected from one of the following:

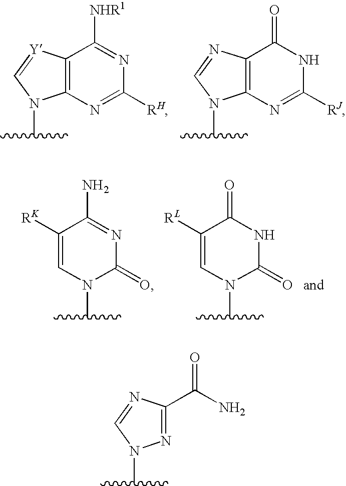

wherein: R^{H} can be hydrogen or halogen; R^{I} can be hydrogen, an optionally substituted C₁₋₄alkyl, or an optionally substituted C₃₋₈cycloalkyl; R^{J} can be hydrogen or amino; R^{K} can be hydrogen or halogen; R^{L} can be hydrogen or an optionally substituted C₁₋₄alkyl; and Y′ can be N (nitrogen) or CR^{M}, wherein R^{M} hydrogen, halogen or an optionally substituted C₁₋₄-alkyl.

In some embodiments, the compound of Formula (II) can be an anti-neoplastic agent. In other embodiments, the compound of Formula (II) can be an anti-viral agent. In still other embodiments, the compound of Formula (II) can be an anti-parasitic agent. Examples of compounds of Formula (II) include, but are not limited to, the following:

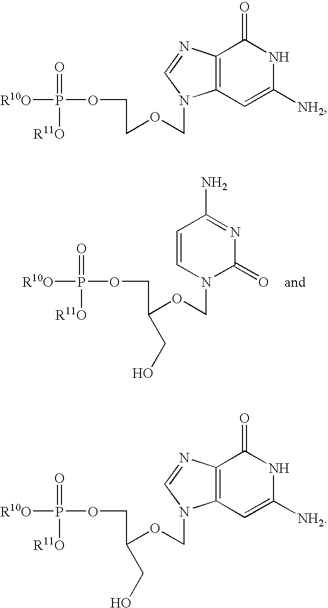

An embodiment disclosed herein relates to a compound of Formula (III) or a pharmaceutically acceptable salt, prodrug or prodrug ester thereof:

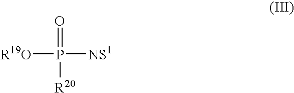

wherein: NS¹ can be a nucleoside attached to the phosphorus via the oxygen bonded to the 5′-carbon; R¹⁹ can be

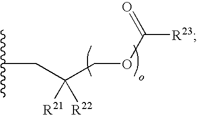

R²⁰ can be a biolabile group linked to the phosphorus that is removed from the phosphorus after the compound of Formula (III) has penetrated a cell; wherein R²¹ and R²² can be each independently —C≡N or an optionally substituted substituent selected from C₁₋₈ organylcarbonyl, C₁₋₈ alkoxycarbonyl and C₁₋₈ organylaminocarbonyl; R²³ can be hydrogen or an optionally substituted C₁₋₄-alkyl; and o can be 1 or 2. In an embodiment, o can be 1. In another embodiment, o can be 2.

Example of R¹⁹ groups, include but are not limited to the following:

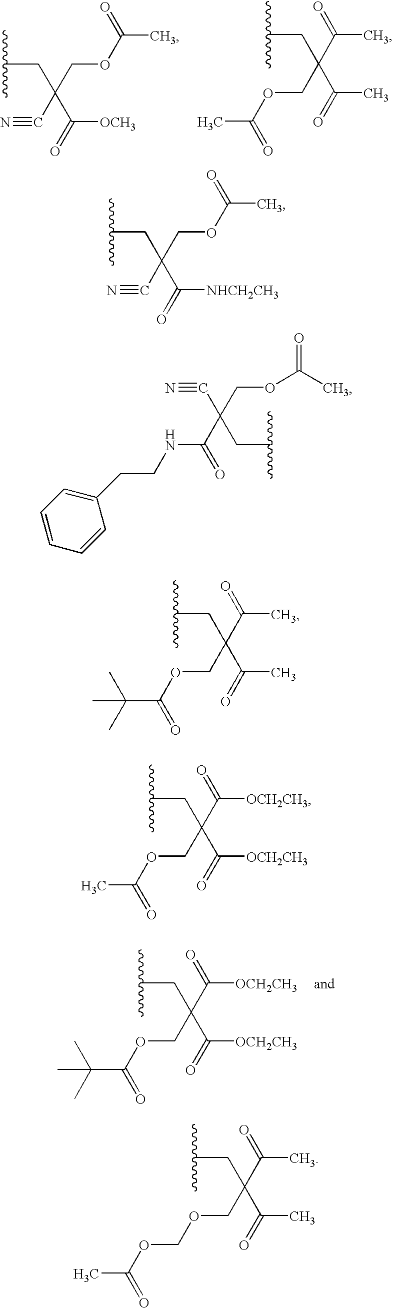

In an embodiment, R¹⁹ can be

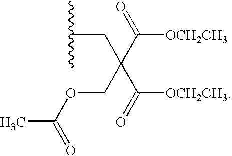

In another embodiment, R¹⁹ can be

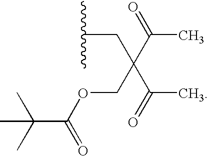

In still another embodiment, R¹⁹ can be

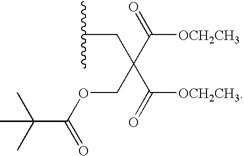

In yet still another embodiment, R¹⁹ can be

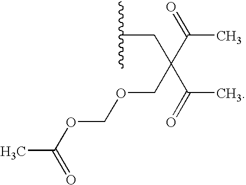

In some embodiments, R¹⁹ can be

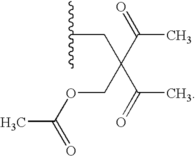

In an embodiment, both R¹⁹ and R²⁰ can be the same group. In some embodiments, both R¹⁹ and R²⁰ can be

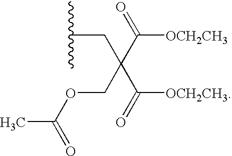

In some embodiments, both R¹⁹ and R²⁰ can be

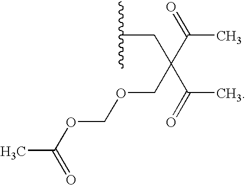

In some embodiments, NS¹ can be selected from adenosine, guanosine, 5-methyluridine, uridine, cytidine and derivatives thereof. In an embodiment, NS¹ can have the structure of Formula (IV).

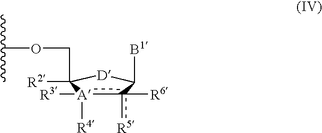

wherein: [symbol] can be a double or single bond; A′ can be selected from C (carbon), O (oxygen) and S (sulfur); B^{1′} can be an optionally substituted heterocyclic base or a derivative thereof; D′ can be C═CH₂ or O; R^{2′} can be selected from hydrogen, azido, —CN, an optionally substituted C₁₋₄ alkyl and an optionally substituted C₁₋₄ alkoxy; R^{3′} can be absent or selected from hydrogen, halogen, hydroxy and an optionally substituted C₁₋₄ alkyl; R^{4′} can be absent or selected from hydrogen, halogen, azido, amino and hydroxy; R^{5′} can be selected from hydrogen, halogen, hydroxy, —CN, —NC, an optionally substituted C₁₋₄ alkyl and an optionally substituted C₁₋₄ alkoxy; and R^{6′} can be absent or selected from hydrogen, halogen, hydroxy, —CN, —NC, an optionally substituted C₁₋₄ alkyl, an optionally substituted haloalkyl and an optionally substituted hydroxyalkyl, or when the bond to R^{5′} indicated by [symbol] is a double bond, then R^{5′} a C₁₋₄ alkenyl and R^{6′} is absent.

In another embodiment, NS¹ can have the structure of Formula (V).

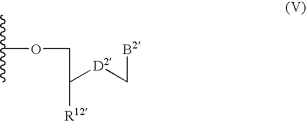

wherein: B^{2′} can be an optionally substituted heterocyclic base or a derivative thereof, D^{2′} can be O (oxygen) or —CH₂—; and R^{12′} can be hydrogen or —(CH₂)—OH.

In some embodiments, the 2,2-disubstituted-acyl(oxyalkyl) groups disclosed herein, such as

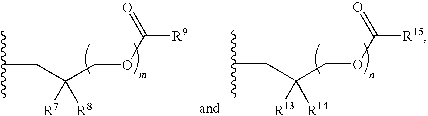

are linked to polynucleotide, oligonucleotide, or an analog thereof.

As used herein, the term “polynucleotide” refer to a polymeric compound made up of any number of covalently bonded nucleotide monomers. Examples of polynucleotides include, but are not limited to, DNA, RNA, oligonucleotides, hybrids of RNA, hybrids of DNA, ribozymes, antisense molecules (e.g., siRNA, miRNA, shRNA, piRNA, and the like), decoy nucleic acids, and the like.

With respect to DNA and RNA, in an embodiment, the DNA or RNA can be single stranded. In another embodiment, the DNA or RNA can be double-stranded. In still another embodiment, the DNA or RNA can be triple-stranded. As used herein, a double-stranded polynucleotide comprises a first single-stranded polynucleotide and a second single-stranded polynucleotide in which at least a portion of the first single-stranded polynucleotide is capable of hybridizing with at least a portion of the second single-stranded polynucleotide. It is not necessary that the first and the second single-stranded polynucleotides in a double-stranded polynucleotide or duplex are 100% complementary. The first single-stranded polynucleotide has to be complementary to a certain degree with at least a portion of the second single-stranded polynucleotide. The percentage of (overall) complementarity of two strands of polynucleotides is preferably at least 50%, preferably at least 70%, or more preferably at least 90%. The term “double stranded” also includes polynucleotide hairpin constructs, such as short-hairpins. The term “double stranded” also includes duplex polynucleotide (or short-hairpins) with an overhang. In other words, the double stranded polynucleotides or duplexes not need to be 100% double stranded in the strict sense.

Oligonucleotides are typically made up of a relatively small number of nucleotide monomers. In some embodiments, the oligonucleotide has no more than 30 nucleic acid molecules. In other embodiments, the oligonucleotide has 5-10 nucleic acid molecules. In other embodiments, the oligonucleotide has 10-20 nucleic acid molecules. In still other embodiments, the oligonucleotide has 20-30 nucleic acid molecules.

In some embodiments, the polynucleotides and oligonucleotides can be linear. In other embodiments, the polynucleotides and oligonucleotides may be circular. The polynucleotides and oligonucleotides described herein can include DNA, RNA or a hybrid thereof, where the nucleic acid may contain combination of deoxyribo- and ribo-nucleotides, and combination of bases including uracil, adenine, thymine, cytosine, guanine, inosine, xanthine, thypoxanthine, isocysteine, isoguanine, and the like. The polynucleotides and oligonucleotides can include mixtures of naturally occurring nucleotides and modified nucleotides having non-naturally-occurring portions which function similarly. Alternatively, mixtures of different modified nucleotides, and mixtures of naturally occurring nucleotides can be used. Modified or substituted polynucleotide can be advantageous over native forms because of desirable properties such as, for example, enhanced cellular uptake, enhanced binding ability to target, improved pharmacokinetics, and increased stability in the presence of nucleases.

As used herein, the term “ribozyme” is an abbreviation for “ribonucleic acid enzyme,” also sometimes known as “RNA enzyme” or “catalytic RNA,” and refer to a class of RNA molecules capable of catalyzing a chemical reaction. Many natural ribozymes catalyze either the hydrolysis of one of their own phosphodiester bonds, or the hydrolysis of bonds in other RNAs. They have also been found to catalyze the aminotransferase activity of the ribosome. Some ribozymes may play an important role as therapeutic agents, as enzymes which tailor defined RNA sequences, as biosensors, and for applications in functional genomics and gene discovery.

As used herein, the term “siRNA” is an abbreviation for “short interfering RNA,” also sometimes known as “small interfering RNA” or “silencing RNA,” and refers to a class of about 19-25 nucleotide-long double-stranded RNA molecules in eukaryotes that are involved in the RNA interference (RNAi) pathway that results in post-transcriptional sequence-specific gene silencing. After being processed by the RNAase III enzyme Dicer, siRNAs can hybridize to cognate mRNAs having a sequence homologous to the siRNA sequence and induce mRNA cleavage and degradation.

As used herein, the term “miRNA” is an abbreviation for “microRNA,” and refers to a class of about 21-25 nucleotide-long single-stranded RNA molecules, which plays a role in regulating gene expression. miRNAs are non-coding RNAs that are encoded by genes from whose DNA they are transcribed. Instead of being translated into protein, each primary transcript (a pri-miRNA), which may have a length of greater than 100 nucleotides, is processed into a short stem-loop structure called a pre-miRNA. Pre-miRNAs usually have a length of 50-90 nucleotides, particularly 60-80 nucleotides, and are processed into functional miRNAs. Mature miRNAs are capable of causing post-transcriptional silencing of target genes which have complete or partially complementary sequences to the miRNAs. Preferably, the regions of complementarity are at least 8 to 10 nucleotides long.

As used herein, the term “shRNA” is an abbreviation for “small hairpin RNA,” also sometimes known as “short hairpin RNA.” shRNA is a sequence of RNA that contains a sense sequence, an antisense sequence, and a short loop sequence between the sense and antisense sequences. Because of the complementarity of the sense and antisense sequences, shRNA molecules tend to form hairpin-shaped double-stranded RNA (dsRNA). shRNAs are processed by the RNAase III enzyme Dicer into siRNA which then get incorporated into the RNA-induced silencing complex (RISC) to silence gene expression via RNA interference.

As used herein, the term “piRNA” is an abbreviation for “Piwi-interacting RNA (piRNA),” and refers to a class of small RNA molecules that is expressed in mammalian testes and somatic cells and forms RNA-protein complexes with Piwi proteins. The Piwi proteins are part of a family of proteins called the argonautes, which are active in the testes of mammals and are required for germ-cell and stem-cell development in invertebrates. piRNA has a role in RNA silencing of retrotransposons and other genetic elements in germ line cells via the formation of an RNA-induced silencing complex (RISC). piRNAs are short stretches of RNAs with a typical length of 25-33 nucleotides, making them distinct entities from miRNAs and siRNAs.

As used herein, the term “decoy nucleic acids” refers to a class of nucleic acids that resembles a natural nucleic acid, but is modified to inhibit or interrupt the activity of the natural nucleic acid. A non-limiting list of decoy nucleic acids includes decoy RNA and decoy DNA. For instance, a decoy RNA can mimic the natural binding domain for a ligand, compete with the natural binding target for the binding of a specific ligand, and thereby prevent the natural binding target from binding the specific ligand. A decoy DNA which contains the specific sequence recognized by a transcription factor can compete with the natural binding target sequence for the binding of the transcription factor and thus block transcription.

In some embodiments, the 2,2-disubstituted-acyl(oxyalkyl) groups described herein are present in polynucleotides that have a length of about 5 to about 10 nucleotides, about 10 to about 15 nucleotides, about 15 to about 20 nucleotides, about 20 to about 25 nucleotides, about 25 to about 30 nucleotides, about 30 to about 35 nucleotides, about 35 to about 40 nucleotides, about 40 to about 45 nucleotides, about 45 to about 50 nucleotides, about 55 to about 60 nucleotides, about 60 to about 65 nucleotides, about 65 to about 70 nucleotides, about 70 to about 75 nucleotides, about 75 to about 80 nucleotides, about 80 to about 85 nucleotides, about 85 to about 90 nucleotides, about 90 to about 95 nucleotides, about 95 to about 100 nucleotides, about 105 to about 110 nucleotides, about 110 to about 130 nucleotides, about 130 to about 150 nucleotides, about 150 to about 170 nucleotides, about 170 to about 190 nucleotides, about 190 to about 210 nucleotides, or longer, or any number in between, including fall length genes or RNA transcripts thereof. In some embodiments, the polynucleotide can have, for example, a length of about 18 to about 100 nucleotides, preferably from about 18 to about 80 nucleotides, more preferably from about 18 to about 90 nucleotides, most preferably from about 19 to about 25 nucleotides, particularly 19, 20, 21, 22, 23, 24, or 25 nucleotides.

The 2,2-disubstituted-acyl(oxyalkyl) groups disclosed herein can be present within only one strand or in both strands of the polynucleotide. In some embodiments, only one 2,2-disubstituted-acyl(oxyalkyl) group disclosed herein is present within a polynucleotide. In other embodiments, a plurality of 2,2-disubstituted-acyl(oxyalkyl) groups disclosed herein is present within a polynucleotide. In some embodiments, a 2,2-disubstituted-acyl(oxyalkyl) group disclosed herein is present on every phosphate group of a polynucleotide. In some embodiments, a 2,2-disubstituted-acyl(oxyalkyl) group disclosed herein is present on every other phosphate group of a polynucleotide. In some embodiments, a plurality of 2,2-disubstituted-acyl(oxyalkyl) groups disclosed herein is present on 5-100% of the phosphate groups of a polynucleotide, more preferably 10-70% of the phosphate groups of a polynucleotide, yet more preferably 15-50% of the phosphate groups of a polynucleotide, most preferably 20-40% of the phosphate groups of a polynucleotide.

The skilled artisan will appreciate the polynucleotides (e.g., oligonucleotides, dsDNA, ssDNA, dsRNA, ribozyme, siRNA, miRNA, shRNA, piRNA, decoy nucleic acids, and the like) are not limited by any particular sequence. In some embodiments, the polynucleotides can comprise coding sequence. In some embodiments, the polynucleotides can comprise noncoding sequence, such as regulatory sequence, including a promoter sequence or a promoter-enhancer combination. The polynucleotides can be of any length and can be used in different application such as gene therapy, modulation of gene expression, and gene detection. Polynucleotides also can be useful for diagnostics, therapeutics, prophylaxis, and research can be used in the methods and compounds disclosed herein.

Non-limiting examples of ribozyme, siRNA, shRNA, miRNA, and piRNA molecules useful in the embodiments described herein include those disclosed in databases such as Riboapt DB (http://mcbc.usm.edu/riboaptDB/), siRecords (http://siRecords.umn.edu/siRecords), siRNAdb (http://sima.sbc.su.se/), RNAi Codex database (http://codex.cshl.edu/scripts/newmain.pl), shRNA Clone Library (http://cgap.nci.nih.gov/RNAi/RNAi2), miRBase (http://microrna.sanger.ac.uk/), piRNABank (http://pirnabank.ibab.ac.in/), and RNAdb (http://research.imb.uq.edu.au/rnadb/). Although exemplary noncoding RNA including ribozyme, siRNA, shRNA, miRNA, and piRNA molecules are described herein, the skilled artisan will readily appreciate that the compounds and methods disclosed herein are useful for any polynucleotides such as ribozymes, siRNAs, miRNAs, shRNAs, piRNA, dsRNAs, RNAi's, and oligonucleotides now known or discovered in the future. In a general sense, the operability of the methods and compounds disclosed herein is not dependent on the sequence or function of the polynucleotides. Rather, the disclosed methods and compounds are useful for delivering polynucleotides into a cell.

Without asking to be bound by any particular theory, it is believed that neutralizing the charge on the phosphate group facilitates the penetration of the cell membrane by compounds of Formulae (I), (II), (III), (IV) and (V) by making the compound more lipophilic. Furthermore, it is believed that the 2,2-disubstituted-acyl(oxyalkyl) groups, such as

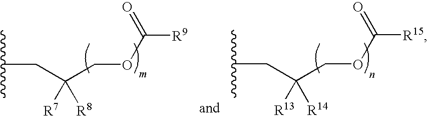

attached to the phosphate impart increased plasma stability to compounds of Formulae (I), (II), (III), (IV) and (V) by inhibiting the degradation of the compound. Once inside the cell, the 2,2-disubstituted-acyl(oxyalkyl) groups attached to the phosphate can be easily removed by esterases via enzymatic hydrolysis of the acyl group. The remaining portions of the group on the phosphate can then be removed by elimination. The general reaction scheme is shown below in Scheme 1. Upon removal of the 2,2-disubstituted-acyl(oxyalkyl) group, the resulting nucleotide analog possesses a monophosphate. Thus, the necessity of an initial intracellular phosphorylation is no longer a prerequisite to obtaining the biologically active phosphorylated form.

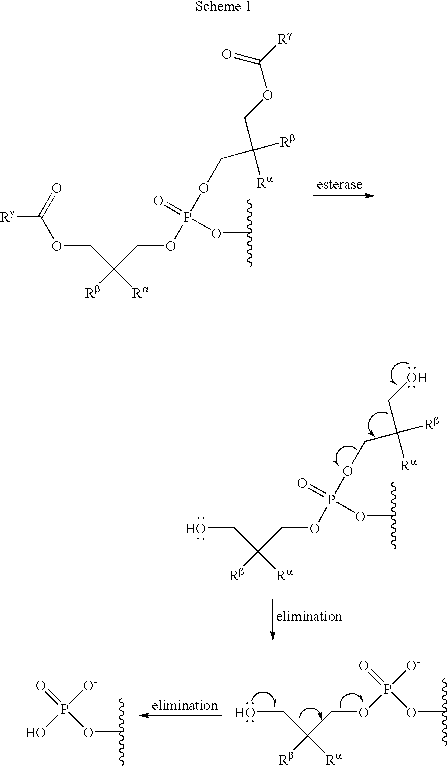

A further advantage of the 2,2-disubstituted-acyl(oxyalkyl) groups described herein is the rate of elimination of the remaining portion of the 2,2-disubstituted-acyl(oxyalkyl) group is modifiable. Depending upon the identity of the groups on the 2-carbon, shown in Scheme 1 as R^{α} and R^{β}, the first elimination may be adjusted from several seconds to several hours. As a result, the removal of the remaining portion of the 2,2-disubstituted-acyl(oxyalkyl) group can be retarded, if necessary, to enhance cellular uptake but, readily eliminated upon entry into the cell.

### Synthesis

Compounds of Formulae (I) (II), (III), (IV), (V) and those described herein may be prepared in various ways. General synthetic routes to the compounds of Formulae (I) and (II), and the starting materials used to synthesize the compounds of Formulae (I) and (II) are shown in Schemes 2a-2c. The routes shown are illustrative only and are not intended, nor are they to be construed, to limit the scope of the claims in any manner whatsoever. Those skilled in the art will be able to recognize modifications of the disclosed synthesis and to devise alternate routes based on the disclosures herein; all such modifications and alternate routes are within the scope of the claims.

The hydroxy precursors,

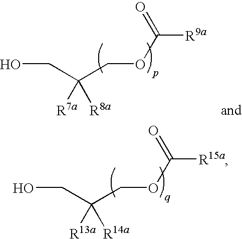

in which R^{7a}, R^{8a}, R^{9a}, R^{13a}, R^{14a}, R^{15a}, p and q are the same as R⁷, R⁸, R⁹, R¹³, R¹⁴, R¹⁵, m and n, respectively, as described herein, of the 2,2-disubstituted-acyl(oxyalkyl) groups can be synthesized according in a manner similar to those described in the following articles. Ora, et al., J. Chem. Soc. Perkin Trans. 2, 2001, 6, 881-5; Poijärvi, P. et al., Helv. Chim. Acta. 2002, 85, 1859-76; Poijärvi, P. et al., Lett. Org. Chem., 2004, 1, 183-88; and Poijärvi, P. et al., Bioconjugate Chem., 2005 16(6), 1564-71, all of which are hereby incorporated by reference in their entireties.

Some embodiments disclosed herein relates to a compound selected from:

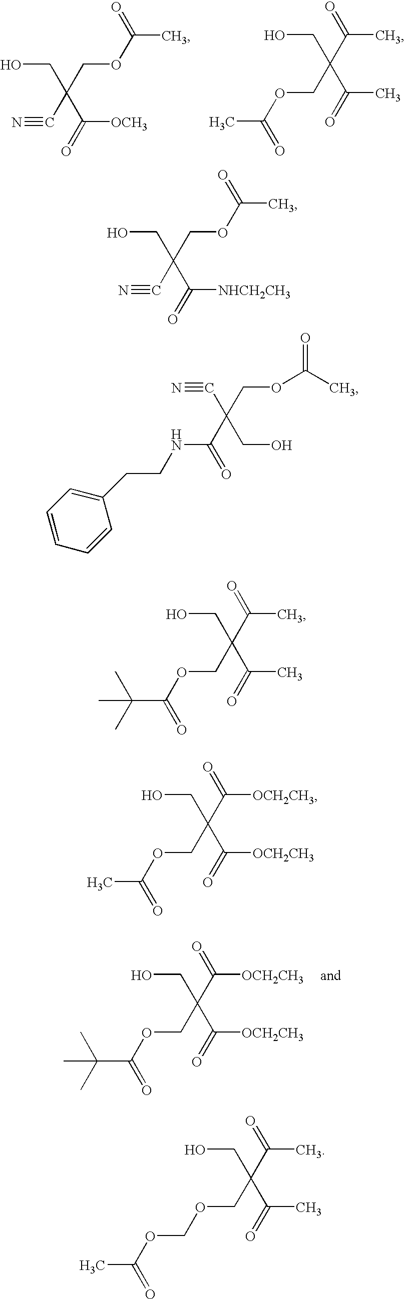

In an embodiment, the compound can be

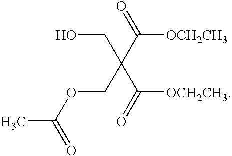

In another embodiment, the compound can be

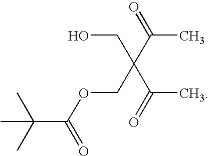

In still another embodiment, the compound can be

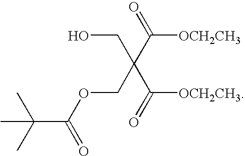

In an embodiment, the compound can be

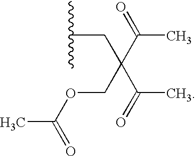

In yet still another embodiment, the compound can be

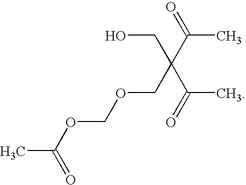

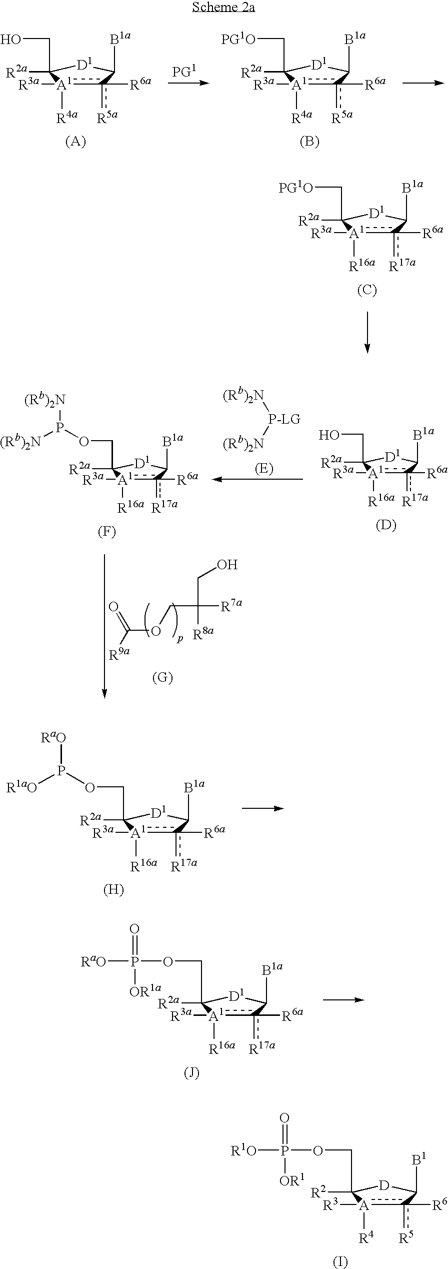

One embodiment disclosed herein relates to a method of synthesizing a compound of Formula (I) that includes the transformations shown in Scheme 2a. In Scheme 2a, A¹, B^{1a}, D¹, R^{2a}, R^{3a}, R^{4a}, R^{5a}, R^{6a}, R^{7a}, R^{8a}, R^{9a} and p can be the same as A, B¹, D, R², R³, R⁴, R⁵, R⁶, R⁷, R⁸, R⁹ and m, respectively, as described above with respect Formula (I). Additionally, in Scheme 2a, R^{a} and R^{1a} can be the same as R¹ as described above with respect Formula (I). R^{16a}, in Scheme 2a, can be absent or selected from hydrogen, halogen, azido, amino, hydroxy, and O-PG², wherein if R^{4a} is not a hydroxy group, then R^{16a} is R^{4a}. In Scheme 2a, R^{17a} can be selected from hydrogen, halogen, hydroxy, —CN, —NC, an optionally substituted C₁₋₄ alkyl, an optionally substituted C₁₋₄ alkoxy and O-PG³, wherein if R^{5a} is not a hydroxy group, then R^{17a} is R^{5a}. In some embodiments, each p can be 1. In other embodiments, each p can be 2. In some embodiments, at least one p can be 1. In an embodiment, at least one p can be 2. In some embodiments, one p can be 1, and the other p, if present, can be 2.

A compound of Formula B can be produced by protecting the oxygen linked to the 5′-carbon of a compound of Formula A with an appropriate protecting group, represented by PG¹. In an embodiment, PG¹ can be a triarylmethyl protecting group. A non-limiting list of triarylmethyl protecting groups are trityl, monomethoxytrityl (MMTr), 4,4′-dimethoxytrityl (DMTr), 4,4′,4″-trimethoxytrityl (TMTr), 4,4′,4″-tris-(benzoyloxy) trityl (TBTr), 4,4′,4″-tris (4,5-dichlorophthalimido) trityl (CPTr), 4,4′,4″-tris (levulinyloxy) trityl (TLTr), p-anisyl-1-naphthylphenylmethyl, di-o-anisyl-1-naphthylmethyl, p-tolyldipheylmethyl, 3-(imidazolylmethyl)-4,4′-dimethoxytrityl, 9-phenylxanthen-9-yl (Pixyl), 9-(p-methoxyphenyl)xanthen-9-yl (Mox), 4-decyloxytrityl, 4-hexadecyloxytrityl, 4,4′-dioctadecyltrityl, 9-(4-octadecyloxyphenyl)xanthen-9-yl, 1,1′-bis-(4-methoxyphenyl)-1′-pyrenylmethyl, 4,4′,4″-tris-(tert-butylphenyl)methyl (TTTr) and 4,4′-di-3,5-hexadienoxytrityl.

In a compound of Formula C, if the R^{4a} of the compound of Formula B is a hydroxy group, the oxygen of R^{4a} can be protected with an appropriate protecting group to form R^{16a}. Likewise, if R^{5a} of the compound of Formula B is a hydroxy group, the oxygen of R^{5a} can be protected with an appropriate protecting group to form R^{17a}. By protecting the oxygens of the hydroxy groups at the 2′ and 3′-positions, undesirable side reactions that may occur during later synthetic transformations can be minimized. In some embodiments, PG² can be a triarylmethyl or levulinoyl protecting group. In some embodiments, PG³ can be a triarylmethyl or levulinoyl protecting group. In an embodiment, PG² and PG³ can be levulinoyl protecting groups.

The protecting group, PG¹, on the oxygen linked to the 5′-carbon can be removed using an appropriate reagent known to those skilled in the art to form a compound of Formula D. Exemplary reagents that can used include an acid and a zinc dihalide (e.g., ZnBr₂). In an embodiment, PG¹ can be removed with acetic acid. In one embodiment, PG¹ can be selectively removed, for example, PG¹ can be removed without removing PG² and/or PG³ if one or both are present.

The 5′-position on the compound of Formula D can be transformed to a phosphoamidite by reacting a compound of Formula E with the —OH attached to 5′-carbon of the compound of Formula D to form a compound of Formula F. In an embodiment, each R^{b} can be independently an optionally substituted alkyl, and LG can be a suitable leaving group. In some embodiments, the leaving group on the compound of Formula E can be a halogen. By selectively removing the protecting group on the oxygen linked to the 5′-carbon, the phosphoamidite can be selectively formed on the 5′-position of the compound. Moreover, any protecting groups at the 2′ and 3′ positions can minimize the addition of the phosphorous group at those positions, thus, reducing the number and/or quantity of side products. Minimizing the number and/or quantity of side products can make the separation and isolation of the compound of Formula F more facile.

A compound of Formula G can be added to the phosphoamidite of the compound of Formula F to form a compound of Formula H. To facilitate the reaction, an activator can be used. An exemplary activator is a tetrazole such as benzylthiotetrazole. The tetrazole can protonate the nitrogen of the phosphoamidite making it susceptible to nucleophilic attack by the compound of Formula G. Additional activators that can be used are disclosed in Nurminen, et al., J. Phys. Org. Chem., 2004, 17, 1-17 and Michalski, J. et al., State of the Art. Chemical Synthesis of Biophosphates and their Analogues via P^{III} Derivatives, Springer Berlin (2004) vol. 232, pages 43-47; which is hereby incorporated by reference for their disclosure of additional activators.

The phosphite of the compound of Formula H can be oxidized to a phosphate to form a compound of Formula J. In an embodiment, the oxidation can be carried out using iodine as the oxidizing agent and water as the oxygen donor.

If the substituent attached to the 3′-position is a protected oxygen on the compound of Formula J, the protecting group, PG², can be removed, and if the substituent attached to the 2′-position is a protected oxygen on the compound of Formula J, the protecting group, PG³, can be removed to form the compound of Formula (I) as described herein. In an embodiment, when PG² and PG³ are the levulinoyl group(s), the levulinoyl groups can be removed with hydrazinium acetate. If the substituents attached to the 2′- and 3′-positions are not protected oxygens, the compound of Formula J can be a compound of Formula (I).

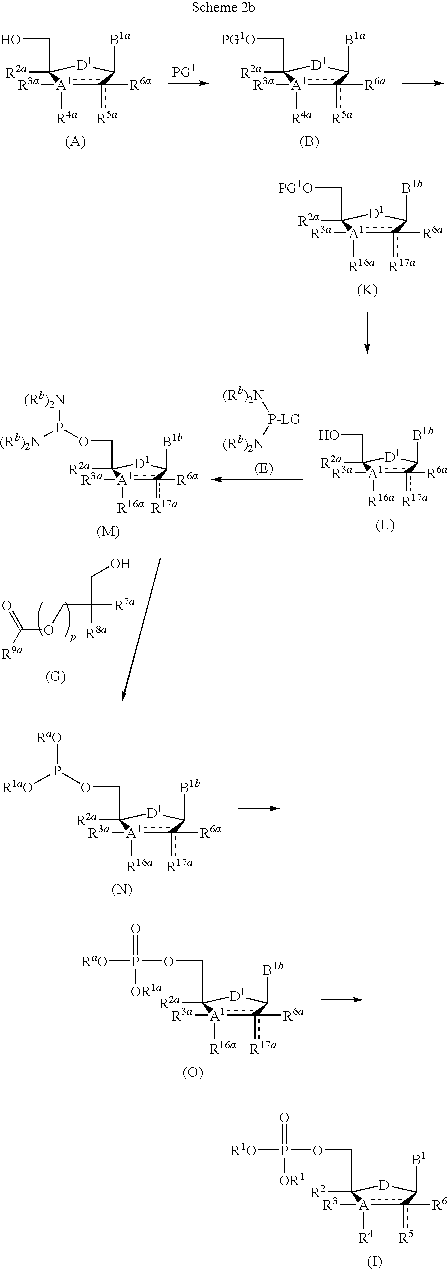

An embodiment disclosed herein relates to a method of synthesizing a compound of Formula (I) that includes the transformations shown in Scheme 2b. In Scheme 2b, A¹, B^{1a}, D¹, R^{2a}, R^{3a}, R^{4a}, R^{5a}, R^{6a}, R^{7a}, R^{8a}, R^{9a} and p can be the same as A, B¹, D, R², R³, R⁴, R⁵, R⁶, R⁷, R⁸, R⁹ and m, respectively, as described above with respect Formula (I). Additionally, in Scheme 2a, R^{a} and R^{1a} can be the same as R¹ as described above with respect Formula (I).

B^{1b} can be an optionally substituted heterocyclic base, an optionally substituted heterocyclic base derivative, an optionally substituted protected heterocyclic base, or an optionally substituted protected heterocyclic base derivative, and if B^{1a} does not have one or more amino groups attached to a ring and/or any —NH groups present in a ring of B^{1a}, then B^{1b} is B^{1a}. In Scheme 2b, R^{16a} can be absent or selected from hydrogen, halogen, azido, amino, hydroxy, and O-PG², wherein if R^{4a} is not a hydroxy group, then R^{16a} is R^{4a}. R^{17a} can be selected from hydrogen, halogen, hydroxy, —CN, —NC, an optionally substituted C₁₋₄ alkyl, an optionally substituted C₁₋₄ alkoxy and O-PG³, wherein if R^{5a} is not a hydroxy group, then R^{17a} is R^{5a}.

As described above, a compound of Formula B can be produced by protecting the oxygen attached to the 5′-carbon of a compound of Formula A with an appropriate protecting group, represented by PG¹. In an embodiment, PG¹ can be a triarylmethyl protecting group. Suitable triarylmethyl protecting groups are described herein. In another embodiment, PG¹ can be a silyl ether. Examples of silyl ether include, but are not limited to trimethylsilyl (TMS), tert-butyldimethylsilyl (TBDMS), triisopropylsilyl (TIPS) and tert-butyldiphenylsilyl (TBDPS).

If the R^{4a} of the compound of Formula B is a hydroxy group, the oxygen of R^{4a} can be protected with an appropriate protecting group to form R^{16a}, and, if R^{5a} of the compound of Formula B is a hydroxy group, the oxygen of R^{5a} can be protected with an appropriate group to form R^{17a}. As stated previously, protecting the oxygens of the hydroxy groups at the 2′ and 3′-positions may minimize undesirable side reactions that may occur during later transformations. In some embodiments, PG² can be a triarylmethyl or levulinoyl protecting group. In some embodiments, PG³ can be a triarylmethyl or levulinoyl protecting group. In an embodiment, PG² and PG³ can be levulinoyl protecting groups.

With respect to a compound of Formula K, if one or more amino groups are attached to B^{1a} and/or a NH group(s) is present in a ring of B^{1a}, the one or more amino groups attached to B^{1a} and/or the nitrogen(s) in the ring of B^{1a} can be protected with one or more suitable protecting groups to form an optionally substituted protected heterocyclic base or derivative thereof. If more than one amino group attached to B^{1a} and/or a NH group(s) is present in a ring of B^{1a} are protected, each protecting group can be the same or different. In some embodiments, the protecting group(s) can be a triarylmethyl protecting group(s).

The protecting group, PG¹, on the oxygen attached to the 5′carbon of the compound of Formula K can be removed using an appropriate reagent known to those skilled in the art to form a compound of Formula L. In one embodiment, PG¹ can be removed with a tetra(alkyl)ammonium halide such as tetra(t-butyl)ammonium fluoride. In an embodiment, PG¹ can be selectively removed without removing one or more protecting groups on B^{1b}. In some embodiments, PG¹ can be selectively removed without removing PG² and/or PG³.

The 5′-position of the compound of Formula L can be transformed to a phosphoamidite by reacting a compound of Formula E with the —OH attached to the 5′-carbon of the compound of Formula L to form a compound of Formula M. The substituents on the compound of Formula E are described herein. By selectively removing the protecting group at the 5′-position, the phosphoamidite can be selectively formed adjacent to the 5′-carbon. Moreover, any protecting groups at the 2′ and 3′ positions can minimize the addition of the phosphorous group at those positions. As a result, the number and/or quantity of side products can be reduced, which in turn, can simplify the separation and isolation of the compound of Formula M.

A compound of Formula G can be added to the phosphoamidite of the compound of Formula M to form a compound of Formula N. As described previously, an activator can be used to assist the reaction.

The phosphite of the compound of Formula N can be oxidizing to a phosphate using an appropriate oxidizing agent and an oxygen source to form a compound of Formula O. In an embodiment, the oxidizing agent can be iodine and the oxygen source can be water.

If the substituent attached to the 3′-position is a protected oxygen on the compound of Formula O, the protecting group represented by PG² can be removed, and if the substituent attached to the 2′-position is a protected oxygen on the compound of Formula O, the protecting group represented by PG³ can be removed. In an embodiment, when PG² and PG³ are levulinoyl groups, the levulinoyl group(s) can be removed with hydrazinium acetate. Additionally, if one or more amino groups are attached to a ring of B^{1b} and/or any nitrogens that are a part of one of the rings of the heterocyclic base are protected, the protecting groups can also be removed to form the compound of Formula (I) as described herein. In some embodiments, the protecting group(s) on B^{1b} can be removed with an acid such as acetic acid or a zinc dihalide such as ZnBr₂. In an embodiment, the protecting group(s) on B^{1b} can be selectively removed such that they can be removed without removing PG² or PG³. In another embodiment, PG² and PG³ can be selectively removed, for example, by removing PG² and/or PG³ without removing one or more protecting groups on B^{1b} If the substituent attached to the 2′-position, the substituent attached to the 3′-position, and the heterocyclic base are not protected, the compound of Formula O is a compound of Formula (I).

The methods of synthesis described above in Schemes 2a and 2b can be used to synthesize any protected nucleotide analogs of Formula (I) and any embodiments of Formula (I) described herein.

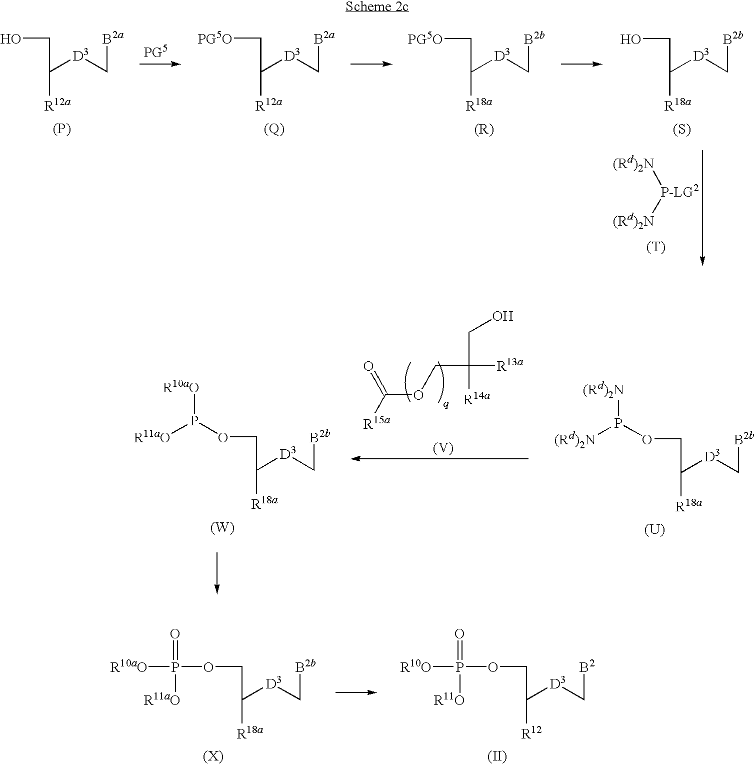

An embodiment disclosed herein relates to a method of synthesizing a compound of Formula (II) that includes the transformations shown in Scheme 2c. In Scheme 2c, D³, R^{10a}, R^{11a}, R^{12a}, R^{13a}, R^{14a}, R^{15a} and q can be the same as D², R¹⁰, R¹¹, R¹², R¹³, R¹⁴, R¹⁵ and n, respectively, as described above with respect Formula (II). In an embodiment, each q can be independently 1. In another embodiment, each q can be 2. In some embodiments, at least one q can be 1. In an embodiment, at least one q can be 2. In some embodiments, one q can be 1, and the other q, if present, can be 2.

In Scheme 2c, B^{2a} can be an optionally substituted heterocyclic base, an optionally substituted heterocyclic base derivative, an optionally substituted protected heterocyclic base, or an optionally substituted protected heterocyclic base derivative, and if B^{2a} does not have one or more amino groups attached to a ring and/or any —NH groups present in a ring of B^{2a}, then B^{2b} is B^{2a}. R^{18a} can be selected from hydrogen, —(CH₂)—OH and —(CH₂)—OPG⁶, wherein if R^{12a} is not —(CH₂)—OH, then R^{18a} is R^{12a}.

A compound of Formula Q can be formed by protecting the hydroxy of a compound of Formula P with an appropriate protecting group, represented by PG⁵. In an embodiment, PG⁵ can be a triarylmethyl protecting group. In another embodiment, PG⁵ can be a silyl ether. Suitable triarylmethyl and silyl ether protecting groups are described herein.

If the R^{12a} of the compound of Formula Q is —(CH₂)—OH, the oxygen of R^{12a} can be protected with an appropriate protecting group to form R^{18a}, wherein when R^{12a} is not —(CH₂)—OH, then R^{18a} is R^{12a}. It may be desirable to protect the oxygen of the hydroxy group in R^{12a} to minimize undesirable side reactions that may occur during later transformations. In an embodiment, R^{18a} can be —(CH₂)—OPG⁶, wherein PG⁶ can be a triarylmethyl or levulinoyl protecting group.

If one or more amino groups are attached to B^{2a} and/or a NH group(s) is present in a ring of B^{2a}, the one or more amino groups attached to B^{2a} and/or nitrogen(s) of B^{2a} can be protected with one or more suitable protecting groups. If more than one amino group attached to B^{2a} and/or a NH group(s) is present in a ring of B^{2a} is protected, each protecting group can be the same or different. In some embodiments, the protecting group(s) can be a triarylmethyl protecting group(s).

The protecting group, PG⁵, can be removed using an appropriate reagent to form a compound of Formula S. In one embodiment, PG⁵ can be removed with a tetra(alkyl)ammonium halide such as tetra(t-butyl)ammonium fluoride. In an embodiment, PG⁵ can be selectively removed without removing PG⁶. In some embodiments, PG⁵ can be selectively removed without removing one or more protecting groups on B^{2b}.

The hydroxy on the compound of Formula S can be transformed to a phosphoamidite by reacting a compound of Formula T with the hydroxy group of the compound of Formula S to form a compound of Formula U. The substituents on the compound of Formula T, each R^{d}, can independently be an optionally substituted C₁₋₄ alkyl.

By selectively removing the PG⁵ in an previous reaction, the phosphoamidite can be selectively formed at one position. Moreover, by protecting any other hydroxy groups, the addition of the phosphorous group to those positions can be minimized, thus, reducing the number and/or quantity of side products. This can make separation and isolation of the compound of Formula U more facile.

A compound of Formula V can be added to the phosphoamidite of the compound of Formula U to form a compound of Formula W. To facilitate the addition, an activator can be used.

The phosphite of the compound of Formula W can be oxidizing to a phosphate using an appropriate oxidizing agent and an oxygen source to form a compound of Formula W. In an embodiment, the oxidizing agent can be iodine and the oxygen source can be water.

If the oxygen of R^{18a} is protected on the compound of Formula W, the protecting group represented by PG⁶ can be removed, and if one or more amino groups are attached to B^{2b} and/or any nitrogens that are a part of one of the rings of the heterocyclic base are protected, the protecting groups can also be removed to form the compound of Formula (II) as described herein. In some embodiments, the protecting group(s) on B^{2b} can be removed with an acid such as acetic acid or a zinc dihalide such as ZnBr₂. In an embodiment, if PG⁶ is present and a levulinoyl group, PG⁶ can be removed using hydrazinium acetate. In some embodiments, the protecting group(s) on B^{2b} can be selectively removed (e.g., the protecting group(s) on B^{2b} can be removed without removing PG⁶). In another embodiment, PG⁶ can be selectively removed such that PG⁶ can be removed without removing one or more protecting groups on B^{2b}. If there are no protecting groups present on the compound of Formula X, then the compound of Formula X can be a compound of Formula (II).

The methods of synthesis described above in Scheme 2c can be used to synthesize any protected nucleotide analogs of Formula (II) and any embodiments of Formula (II) describe herein.

Various protecting groups can be present on the compounds in Schemes 2a to 2c. One benefit of having these protecting groups is that the addition of one or more compounds can be directed to certain positions of another compound(s). Furthermore, as previously discussed, the protecting groups can block undesirable side reactions that may occur during later synthetic transformations. Minimization of unwanted side compound can make the separation and isolation of the desired compound(s) more facile.

An embodiments disclosed herein relates to a method that can include reacting a first reactant, wherein the first reactant can include a nucleoside with a phosphoamidite attached to the 5′-carbon or a protected nucleoside derivative with a phosphoamidite attached to the 5′-carbon, with a second reactant that can include a compound of Formula (G) having the structure

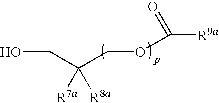

to form a compound having the structure

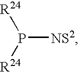

wherein at least one R²⁴ has the formula

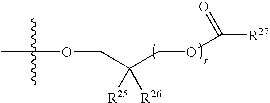

wherein R^{7a}, R^{8a}, R²⁵ and R²⁶ can be each independently C≡N or an optionally substituted substituent selected from C₁₋₈ organylcarbonyl, C₁₋₈ alkoxycarbonyl and C₁₋₈ organylaminocarbonyl; R^{9a} and R²⁷ can be each independently hydrogen or an optionally substituted C₁₋₄-alkyl; p can be 1 or 2; r can be 1 or 2; NS² can be a nucleoside or a protected nucleoside derivative; and the other R²⁴ can be a biolabile group. In an embodiment, the phosphoamidite can have the structure:

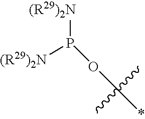

wherein each R²⁹ can independently be an optionally substituted C₁₋₄ alkyl; and the bond denoted with a “*” links the phosphoamidite to the 5′-carbon of the nucleoside or protected nucleoside derivative.

Another embodiment disclosed herein relates to a method that can include oxidizing the phosphorus in a compound having the structure

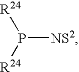

wherein at least one R²⁴ has the formula

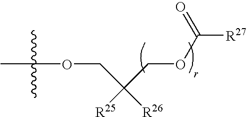

wherein R²⁵ and R²⁶ can be each independently —C≡N or an optionally substituted substituent selected from C₁₋₈ organylcarbonyl, C₁₋₈ alkoxycarbonyl and C₁₋₈ organylaminocarbonyl; R²⁷ can be hydrogen or an optionally substituted C₁₋₄-alkyl; r can be 1 or 2; NS² can be a nucleoside or a protected nucleoside derivative; and the other R²⁴ is a biolabile group, to phosphorus (V).

Examples of suitable R²⁴ groups include, but are not limited to,

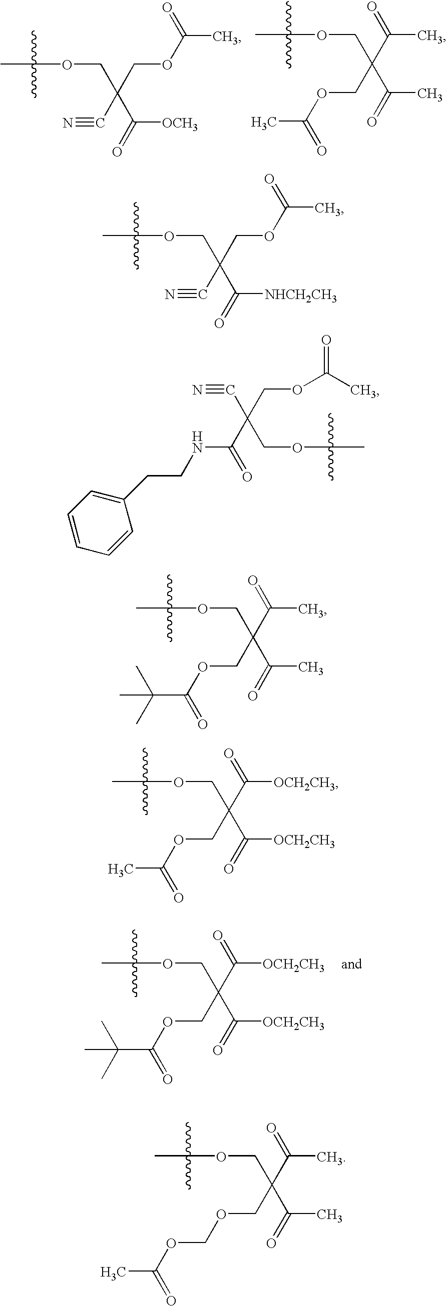

In some embodiments, both R groups can be the same. For example, in an embodiment, both R²⁴ groups can be

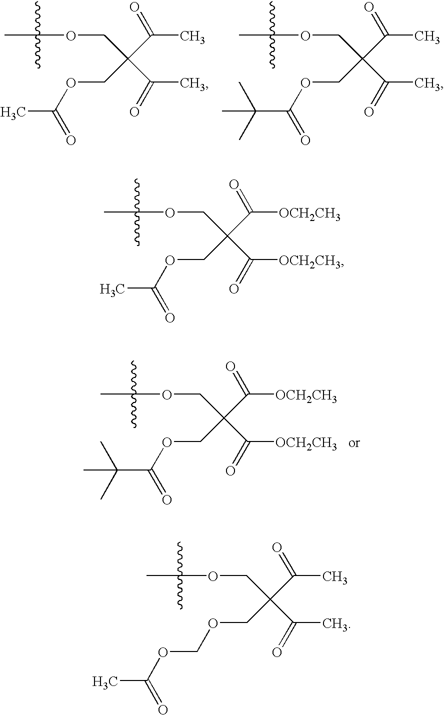

In another embodiment, the R²⁴ groups can be different.

The phosphorus in a compound having the structure

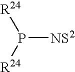

can be oxidized to phosphorus (V) using method known to those skilled in the art, such as an appropriate oxidizing agent and an oxygen source. In an embodiment, the oxidizing agent can be iodine and the oxygen source can be water.

Still another embodiment disclosed herein relates to a method comprising reacting a first reactant, wherein the first reactant can include a phosphite, with a second reactant that can include a compound of Formula (G) having the structure

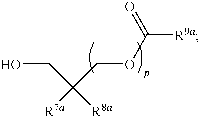

and a third reactant that can include a nucleoside or a protected nucleoside derivative; to form a compound having the structure

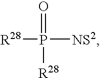

wherein at least one R²⁸ has the formula

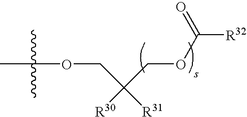

wherein R^{7a}, R^{8a}, R³⁰ and R³¹ can be each independently —C≡N or an optionally substituted substituent selected from C₁₋₈ organylcarbonyl, C₁₋₈ alkoxycarbonyl and C₁₋₈ organylaminocarbonyl; R^{9a} and R³² can be each independently hydrogen or an optionally substituted C₁₋₄-alkyl; p can be 1 or 2; s can be or 2; NS² can be a nucleoside or a protected nucleoside derivative; and the other R²⁸ is a biolabile group. In an embodiment, the phosphite can be diphenylphosphite.

Examples of suitable R²⁸ groups include, but are not limited to,

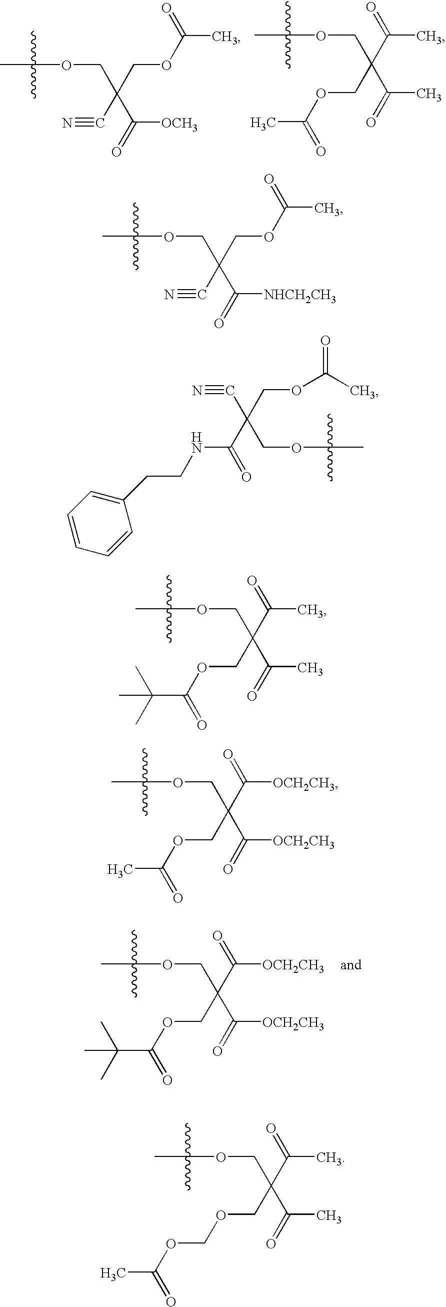

In some embodiments, both R²⁸ groups can be the same. For example, in an embodiment, both R²⁸ groups can be

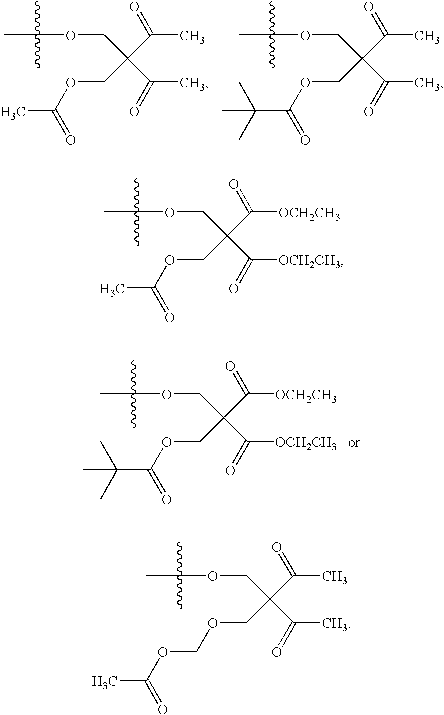

In another embodiment, the R²⁸ groups can be different.

The 2,2-disubstituted-acyl(oxyalkyl) groups disclosed herein, such as

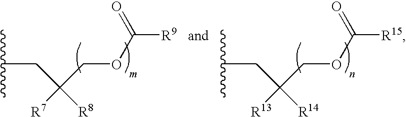

can be introduced into a polynucleotide, an oligonucleotide, or an analog thereof using methods known to those skilled in the art. For example, a variant can be made using the methods described in the schemes herein, such as Scheme 2b, in which the phosphoamidite is formed at the 3′-position instead of the 5′-position. This can be accomplished by protecting the —OH group attached to the 5′-carbon with a suitable protecting group such as a triarylmethyl protecting group described herein while leaving the hydroxy attached to the 3′-position unprotected. The variant can have the following structure:

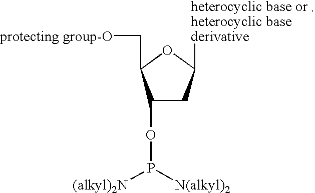

Using an automated DNA synthesizer and methods known to those skilled in the art, the variant and the 2,2-disubstituted-acyl(oxyalkyl) groups disclosed herein can be used to form a polynucleotide (for example, DNA) that incorporates one or more of the 2,2-disubstituted-acyl(oxyalkyl) groups disclosed herein.

### Pharmaceutical Compositions

An embodiment described herein relates to a pharmaceutical composition, comprising a therapeutically effective amount of one or more compounds described herein (e.g., a compound of Formula (I), a compound of Formula (II), a compound of Formula (III), a compound of Formula (IV) and/or a compound of Formula (V)) and a pharmaceutically acceptable carrier, diluent, excipient or combination thereof.

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, intramuscular, intraocular, intranasal, intravenous, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical compositions will generally be tailored to the specific intended route of administration.

The term “physiologically acceptable” defines a carrier, diluent or excipient that does not abrogate the biological activity and properties of the compound.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Suitable routes of administration may, for example, include oral, rectal, topical transmucosal, or intestinal administration; parenteral delivery, including intramuscular, subcutaneous, intravenous, intramedullary injections, as well as intrathecal, direct intraventricular, intraperitoneal, intranasal, intraocular injections or as an aerosol inhalant.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes will be targeted to and taken up selectively by the organ.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

### Methods of Use

One embodiment disclosed herein relates to a method of treating and/or ameliorating a disease or condition by administering to a subject a therapeutically effective amount of one or more compounds described herein, such as a compound of Formula (I), a compound of Formula (II), a compound of Formula (III), a compound of Formula (IV) and/or a compound of Formula (V), or a pharmaceutical composition that includes a compound described herein.

Some embodiments disclosed herein relate to a method of ameliorating or treating a neoplastic disease that can include administering to a subject suffering from the neoplastic disease a therapeutically effective amount of one or more compounds described herein (e.g., a compound of Formula (I), a compound of Formula (II), a compound of Formula (III), a compound of Formula (IV) and/or a compound of Formula (V)) or a pharmaceutical composition that includes one or more compounds described herein. In an embodiment, the neoplastic disease can be cancer. In some embodiments, the neoplastic disease can be a tumor such as a solid tumor. In an embodiment, the neoplastic disease can be leukemia. Exemplary leukemias include, but are not limited to, acute lymphoblastic leukemia (ALL), acute myeloid leukemia (AML) and juvenile myelomonocytic leukemia (JMML).

An embodiment disclosed herein relates to a method of inhibiting the growth of a tumor comprising administering to a subject having the tumor a therapeutically effective amount of one or more compounds described herein or a pharmaceutical composition that includes one or more compounds described herein.

Other embodiments disclosed herein relates to a method of ameliorating or treating a viral infection comprising administering to a subject suffering from the viral infection a therapeutically effective amount of one or more compounds described herein or a pharmaceutical composition that includes one or more compounds described herein. In an embodiment, the viral infection can be caused by a virus selected from an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxyiridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae and/or a Togaviridae. In an embodiment, the viral infection is a hepatitis C viral infection. In another embodiment, the viral infection is a HIV infection.

One embodiment disclosed herein relates to a method of ameliorating or treating a parasitic disease comprising administering to a subject suffering from the parasitic disease a therapeutically effective amount of one or more compounds described herein or a pharmaceutical composition that includes one or more compounds described herein. In an embodiment, the parasite disease can be Chagas' disease.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

The term “therapeutically effective amount” is used to indicate an amount of an active compound, or pharmaceutical agent, that elicits the biological or medicinal response indicated. For example, a therapeutically effective amount of compound can be the amount need to prevent, alleviate or ameliorate symptoms of disease or prolong the survival of the subject being treated This response may occur in a tissue, system, animal or human and includes alleviation of the symptoms of the disease being treated. Determination of a therapeutically effective amount is well within the capability of those skilled in the art, especially in light of the detailed disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. (See e.g., Fingl et al. 1975, in “The Pharmacological Basis of Therapeutics”, which is hereby incorporated herein by reference in its entirety, with particular reference to Ch. 1, p. 1). The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the patient. In some embodiments, the compounds will be administered for a period of continuous therapy, for example for a week or more, or for months or years.

In instances where human dosages for compounds have been established for at least some condition, those same dosages, or dosages that are between about 0.1% and 500%, more preferably between about 25% and 250% of the established human dosage will be used. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Alternatively dosages may be based and calculated upon the surface area of the patient, as understood by those of skill in the art.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

### EXAMPLES

Additional embodiments are disclosed in further detail in the following examples, which are not in any way intended to limit the scope of the claims.

### 1-METHYL 3-ACETOXY-2-CYANO-2-(HYDROXYMETHYL)PROPANOATE (1)

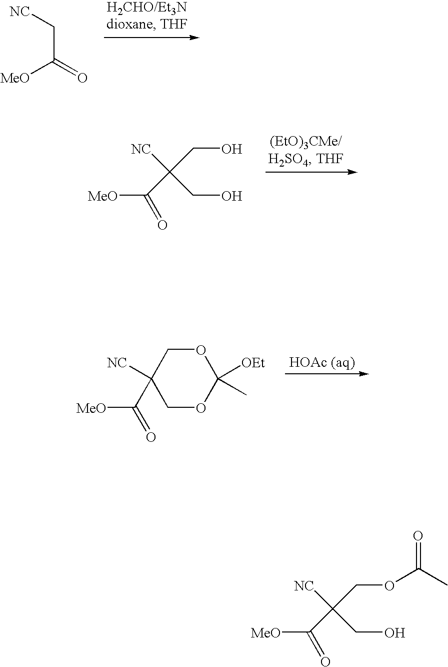

Methyl 2-cyano-3-hydroxy-2-hydroxymethylpropanoate. Formaldehyde (66.7 mmol, 2.0 g) was added as 20% aq solution (10 g) to 1,4-dioxane (30 mL) on an ice-bath. Methyl cyanoacetate (30.3 mmol, 2.12 mL) and Et₃N (0.61 mmol, 0.61 mL of 1 mol L⁻¹ solution in THF) were added and the mixture was stirred for 20 min. Another portion of Et₃N (0.61 mmol) was added and the ice-bath was removed. The mixture was stirred for 1.5 h at room temperature. The mixture was then diluted with water (200 mL) and extracted with benzene (3×50 mL) to remove side products. The aqueous phase was evaporated under reduced pressure at 30° C. to one fourth of the original volume and extracted 5 times with ethyl acetate. The combined extracts were dried over Na₂SO₄ and evaporated to a clear oil. The yield was 72% (4.82 g). The compound was used without characterization to the next step.

Methyl 5-cyano-2-ethoxy-2-methyl-1,3-dioxane-5-carboxylate. Methyl 2-cyano-3-hydroxy-2-hydroxymethylpropanoate (23.3 mmol, 3.7 g) was dissolved in dry THF (8 mL) and triethyl orthoacetate (34.9 mmol, 6.55 mL) was added. A catalytic amount of concentrated sulfuric acid (0.70 mmol, 37 μL) was added and the mixture was stirred over night at room temperature. The mixture was poured into a stirred ice-cold aq NaHCO₃ (5%, 50 mL). The product was extracted into Et₂O (2×50 mL) and the extracts were washed with saturated aq NaCl and dried over Na₂SO₄. The solvent was evaporated and purified by Silica gel chromatography applying a stepwise gradient from 5% ethyl acetate in dichloromethane to pure ethyl acetate. The product was obtained in 42% yield (5.33 g) as a clear oil that started to crystallize. ¹H NMR for the major diastereomer (CDCl₃) 4.34 (d, J=7.0 Hz, 2H, —CH₂O—), 4.03 (d, J=8.5 Hz, 2H, —CH₂O—), 3.84 (s, 3H, OMe), 3.54 (q, J=7.2 Hz, 2H, —CH₂CH₃), 1.55 (s, 3H, —CH₃), 1.25 (t, J=7.2, 3H, —CH₂CH₃). ¹³C NMR for the major diastereomer (CDCl₃) 164.8 (C═O), 117.0 (CN), 111.4 (C2), 62.3 (C4 and C6), 59.1 (—CH₂CH₃), 53.9 (—OCH₃), 42.4 (C5), 22.3 (2-CH₃), 15.0 (CH₂CH₃).

Methyl 3-acetyloxy-2-cyano-2-(hydroxymethyl)propanoate. Methyl 5-cyano-2-ethoxy-2-methyl-1,3-dioxane-5-carboxylate (2.18 mmol, 0.50 g) was dissolved in a mixture of acetic acid and water (4:1, v/v, 20 mL) and the mixture was stirred for 2 h at room temperature, after which the mixture was evaporated to dryness and the residue was coevaporated 3 times with water. The product was purified by Silica gel chromatography, eluting with dichloromethane containing 5% MeOH. The yield was 52% (0.23 g). ¹H NMR (CDCl₃) 4.53 (d, J=11.0 Hz, 1H, —CH₂OAc), 4.50 (d, J=11.0 Hz, 1H, —CH₂OAc), 4.04 (d, J=6.5 Hz, 2H, —CH₂OH), 3.91 (s, 3H, —OMe), 2.90 (t, J=6.5 Hz, —OH), 2.16 (s, 3H, —C(O)CH₃). ¹³C NMR (CDCl₃) 170.4 (C═O), 166.0 (C═O), 116.0 (CN), 63.1 (—CH₂OH), 62.3 (—CH₂OAc), 54.1 (—OMe), 51.0 (C2), 20.6 (—C(O)CH₃).

### 2-CYANO-3-(ETHYLAMINO)-2-(HYDROXYMETHYL)-3-OXOPROPYL ACETATE (2)

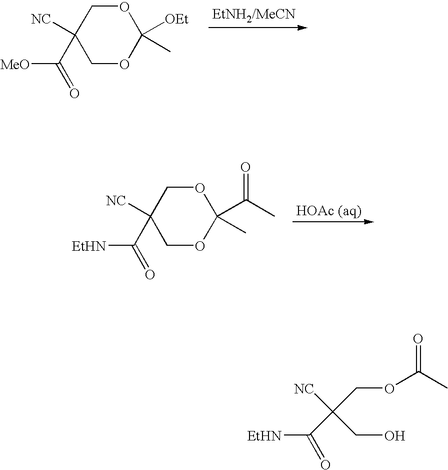

### 2-CYANO-3-(2-PHENYLETHYLAMINO)-2-(HYDROXYMETHYL)-3-OXOPROPYL ACETATE (2b)

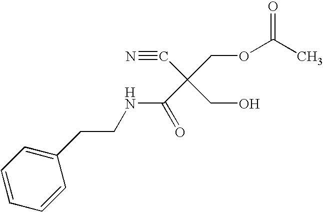

2-cyano-3-(2-phenylethylamino)-2-(hydroxymethyl)-3-oxopropyl acetate was prepared according to the procedure described in Poijärvi, P.; Mäki, E.; Tomperi, J.; Ora, M.; Oivanen, M.; Lönnberg, H., Helve. Chim. Acta. (2002) 85, 1869-1876, which is hereby incorporated by references for the limited purpose of describing the method of synthesizing and purifying 2-cyano-3-(2-phenylethylamino)-2-(hydroxymethyl)-3-oxopropyl acetate.

### 2-ACETYL-2-(HYDROXYMETHYL)-3-OXOBUTYL ACETATE (3)

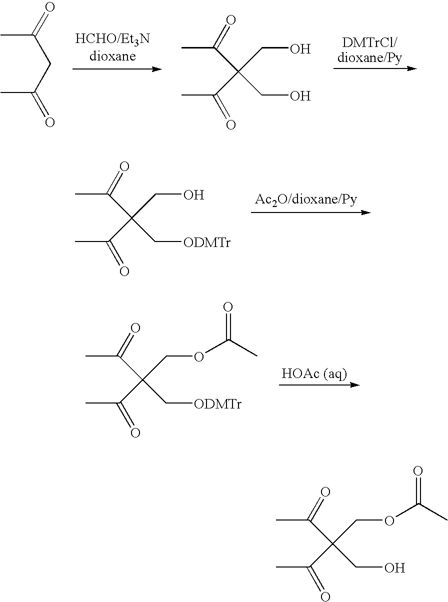

### 2-ACETYL-2-(HYDROXYMETHYL)-3-OXOBUTYL PIVALATE (4)

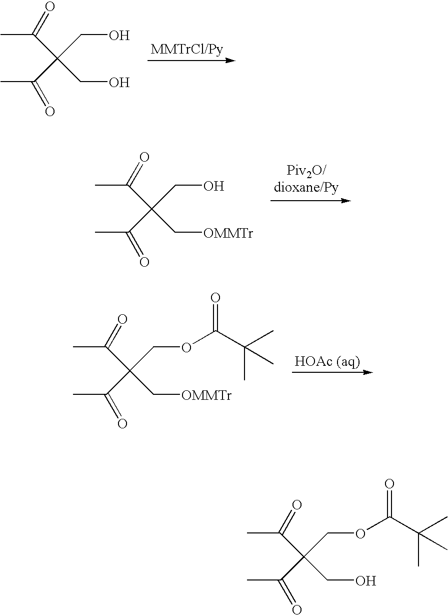

### 2-ACETYL-2-HYDROXYMETHYL-3-OXOBUTYL ACETATE (5)

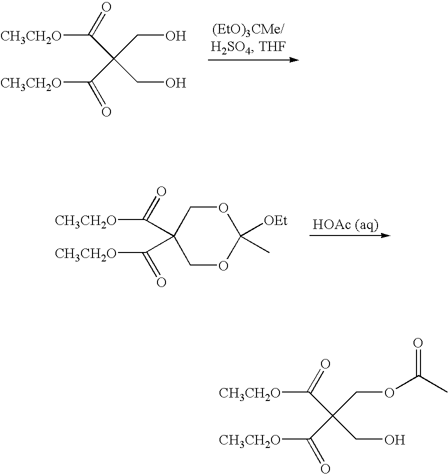

Diethyl 2-ethoxy-2-methyl-1,3-dioxane-5,5-dicarboxylate. Concentrated H₂SO₄ (1.3 mmol; 71 μL) was added to a mixture of diethyl 2,2-bis(hydroxymethyl)malonate (43.5 mmol, 9.6 g) and triethyl orthoacetate (65.2 mmol; 11.9 mL) in dry THF (15 mL). The reaction was allowed to proceed overnight and the mixture was the poured into an ice-cold solution of 5% NaHCO₃ (50 mL). The product was extracted with diethyl ether (2×50 mL), washed with saturated aqueous NaCl (2×50 mL) and dried over Na₂SO₄. The solvent was evaporated and the crude product was purified on a silica gel column eluting with a mixture of dichloromethane and methanol (95:5, v/v). The product was obtained as clear oil in 89% yield (11.3 g). ¹H NMR δ_{H} (500 MHz, CDCl₃): 4.30-4.36 (m, 6H, 4-CH₂, 6-CH₂ and 5-COOCH₂Me), 4.18 (q, J=7.1 Hz, 5-COOCH₂Me), 3.54 (q, J=7.10 Hz, 2H, 2-OCH₂Me), 1.46 (s, 3H, 2-CH₃), 1.32 (t, J=7.10 Hz, 3H, 2-OCH₂Me), 1.27 (t, J=7.1 Hz 3H, 5-COOCH₂Me), 1.26 (t, J=7.1 Hz 3H, 5-COOCH₂Me). ¹³C NMR (500 MHz, CDCl₃): δ=168.0 and 167.0 (5-COOEt), 111.1 (C2), 62.0 and 61.9 (5-COOCH₂Me), 61.6 (C4 and C6), 58.7 (2-OCH₂Me), 52.3 (C5), 22.5 (2-Me), 15.1 (2-OCH₂CH₃), 14.0 and 13.9 (5-COOCH₂CH₃).

Diethyl 2-(acetyloxymethyl)-2-(hydroxymethyl)malonate. Diethyl 2-ethoxy-2-methyl-1,3-dioxane-5,5-dicarboxylate (17.9 mmol; 5.2 g) was dissolved in 80% aqueous acetic acid (30 mL) and left for 2 h at room temperature. The solution was evaporated to dryness and the residue was coevaporated three times with water. The product was purified by silica gel column chromatography eluting with ethyl acetate in dichloromethane (8:92, v/v). The product was obtained as yellowish oil in 75% yield (3.6 g). ¹H NMR δ_{H} (500 MHz, CDCl₃): 4.76 (s, 2H, CH₂OAc), 4.26 (q, J=7.10 Hz, 4H, OCH₂Me), 4.05 (d, J=7.10 Hz, 2H, CH₂OH), 2.72 (t, J=7.1 Hz, 1H, CH₂OH), 2.08 (s, 3H, Ac), 1.27 (t, J=7.10 Hz, 6H, OCH₂CH₃). ¹³C NMR (500 MHz, CDCl₃): δ=170.9 (C═O Ac), 168.1 (2×C═O malonate), 62.3 and 62.2 (CH₂OH and CH₂OAc), 61.9 (2×OCH₂CH₃) δ9.6 (spiro C), 20.7 (CH₃ Ac), 14.0 (2×OCH₂CH₃).

### 2,2-BIS(ETHOXYCARBONYL)-3-HYDROXYPROPYL PIVALATE (6)

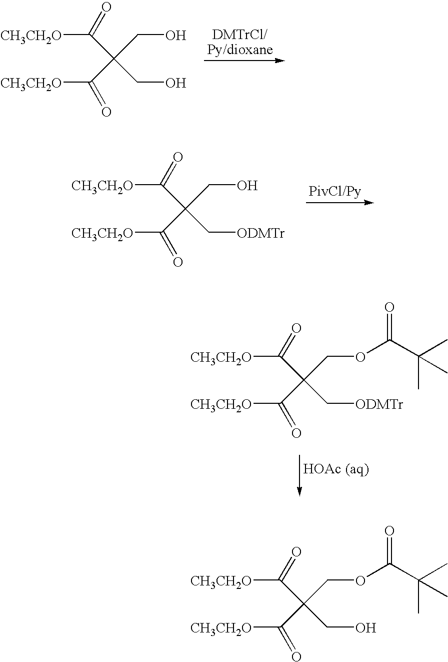

2,2-Bis(ethoxycarbonyl)-3-(4,4′-dimethoxytrityloxy)propyl pivalate. Diethyl 2,2-bis(hydroxymethyl)malonate was reacted with 1 equiv. of 4,4′-dimethoxytrityl chloride in 1,4-dioxane containing 1 equivalent of pyridine. Diethyl 2-(4,4′-dimethoxytrityloxymethyl)-2-(hydroxymethyl)malonate (2.35 g, 4.50 mmol) was acylated with pivaloyl chloride (0.83 mL, 6.75 mmol) in dry MeCN (10 mL) containing 3 equivalent pyridine (1.09 mL, 13.5 mmol). After 3 days at room temperature, the reaction was quenched with MeOH (20 mL) and a conventional CH₂Cl₂/aq HCO₃⁻ workup was carried out. Silica gel chromatography (EtOAc/hexane 1:1, v/v) gave 2.47 g (90%) of the desired product as yellowish syrup. ¹H NMR (CDCl₃, 200 MHz): 7.13-7.39 [m, 9H, (MeO)₂ Tr], 6.81 (d, 4H, [MeO]₂ Tr), 4.71 (s, 2H, CH₂OPiv); 4.15 (q, J=7.1, 4H, OCH₂CH₃); 3.78 [s, 6H, (CH₃O)₂Tr]; 3.67 (s, 2H, CH₂ODMTr); 1.27 (t, J=7.1, 6H, OCH₂CH₃); 1.02 [s, 9H, COC(CH₃)₃].

2,2-Bis(ethoxycarbonyl)-3-hydroxypropyl pivalate. 2,2-Bis(ethoxycarbonyl)-3-(4,4′-dimethoxytrityloxy)propyl pivalate (2.47 g, 4.07 mmol) in a 4:1 mixture of CH₂Cl₂ and MeOH (20 mL) was treated for 4 h at room temperature with TFA (2.00 mL, 26.0 mmol) to remove the dimethoxytrityl group. The mixture was neutralized with pyridine (2.30 mL, 28.6 mmol), subjected to CH₂Cl₂/aq workup and purified by silica gel chromatography (EtOAc/hexane 3:7, v/v) to obtain 1.15 g (93%) of the desired product. ¹H NMR (CDCl₃, 200 MHz): 4.59 (s, 2H, CH₂OPiv); 4.25 (q, J=7.1, 4H, OCH₂CH₃); 4.01 (s, 2H, CH₂OH); 1.28 (t, J=7.1, 6H, OCH₂CH₃); 1.18 [s, 9H, COC(CH₃)₃]. ESI-MS⁺: m/z 305.4 ([MH]⁺), 322.6 ([MNH₄]⁺), 327.6 ([MNa]⁺), 343.5 ([MK]⁺).

### 2′,3′-DI-O-LEVULINOYLRIBAVIRIN (10)

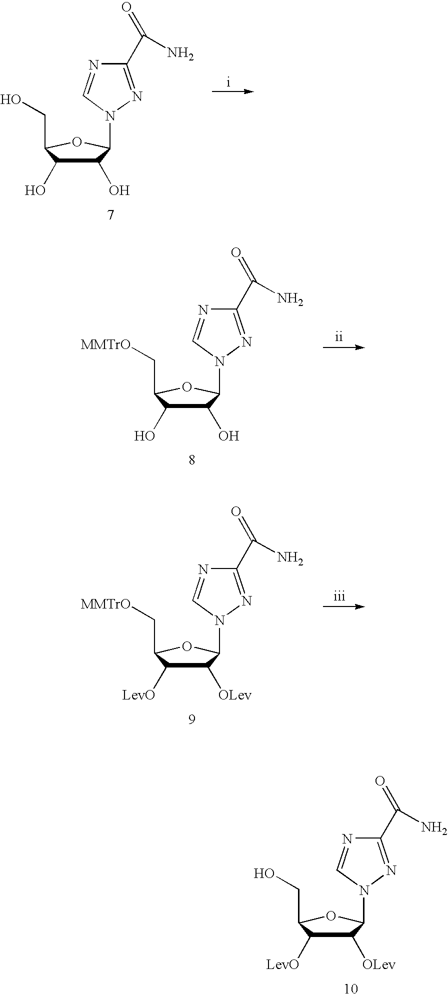

5′-O-(4-Methoxytrityl)ribavirin (8). Ribavirin (compound 7; 8.31 mmol; 2.03 g) was dried by repeated coevaporations from dry pyridine and dissolved in the same solvent (15 mL). 4-Methoxytrityl chloride (8.32 mmol; 2.57 g) was added and the reaction was allowed to proceed overnight. The mixture was evaporated to dryness and the residue was equilibrated between chloroform and water. The organic phase was dried on Na₂SO₄. The crude product was purified by silica gel chromatography using gradient elution from 5 to 10% MeOH in DCM. Yield 68%. ¹H NMR (CDCl₃) δ 8.45 (s, 1H, H5), 7.39-741 (m, 4H, MMTr), 7.27-7.30 (m, 2H, MMTr), 7.21-7.24 (m, 4H, MMTr), 7.15-7.18 (m, 2H, MMTr), 7.09 (br s, 1H, NH), 6.78-6.80 (m, 2H, MMTr), 6.43 (br s, 1H, NH), 5.98 (d, J=3.5 Hz, 1H, H1′), 4.79 (dd, J=3.5 and 4.7 Hz, 1H, H2′), 4.48 (dd, J=4.7 and 5.1, 1H, H3′), 4.31 (m, 1H, H4′), 3.73 (s, 3H, MeO-MMTr), 3.43 (dd, J=10.6 and 2.8 Hz; 1H, H5′), 3.31 (dd, 10.6 and 4.3 Hz, 1H, H5″). ¹³C NMR (CDCl₃) δ 161.3 (C═O), 158.6 (MMTr), 156.5 (C3), 144.6 (MMTr), 144.0 (C5), 136.3 (MMTr), 130.4 (MMTr), 128.3 (MMTr), 127.9 (MMTr), 127.0 (MMTr), 113.2 (MMTr), 92.9 (C1′), 86.7 (MMTr), 84.6 (C4′), 75.3 (C2′), 71.1 (C3′), 63.5 (C5′), 55.2 (MMTr).

2′,3′-Di-O-levulinoyl-5′-O-(4-methoxytrityl)ribavirin (9). Levulinic acid (28.3 mmol; 3.29 g) was dissolved in dry dioxane and the solution was cooled to 0° C. on an ice bath. Dicyclohexylcarbodiimide (14.2 mmol; 2.93 g) was added portionwise during 1 h. Dicyclohexylurea crystallized was removed by filtration. The filtrate and dioxane washing of the precipitate (5 mL) were combined and mixed with the solution of compound 8 (dried on P2O5) in dry pyridine (15 mL). A catalytic amount of 4-dimethylaminopyridine was added and the reaction was allowed to proceed overnight. Volatiles were removed under reduced pressure and the residue was subjected to DCM/aq NaHCO₃ work-up. The organic phase was dried on Na₂SO₄. The crude product (9) was used in the next step. ¹H NMR (CDCl₃) δ 8.36 (s, 1H, H5), 7.42-7.44 (m, 4H, MMTr), 7.23-7.32 (m, 8H, MMTr), 6.78-6.80 (m, 2H, MMTr), 6.66 (br s, 1H, NH), 6.08 (d, J=4.9 Hz, 1H, H1′), 6.00 (dd, J=4.9 and 5.3 Hz, 1H, H2′), 5.69 (br s, 1H, NH), 5.63 (dd, J=4.1 and 5.3, 1H, H3′), 4.40 (m, 1H, H4′), 3.80 (s, 3H, MeO-MMTr), 3.47 (dd, J=10.6 and 2.8 Hz; 1H, H5′), 3.36 (dd, 10.8 and 4.3 Hz, 1H, H5″), 2.76-2.81 (m, 4H, Lev), 2.61-2.67 (m, 4H, Lev), 2.20 (s, 6H, Lev). ¹³C NMR (CDCl₃) δ 206.3 (2×C═O Lev), 171.6 (C═O Lev), 171.3 (C═O Lev), 160.3 (C═O), 158.7 (MMTr), 157.2 (C3), 144.7 (MMTr), 143.7 (C5), 136.0 (MMTr), 130.5 (MMTr), 128.4 (MMTr), 128.0 (MMTr), 127.2 (MMTr), 113.2 (MMTr), 89.9 (C1′), 87.2 (MMTr), 83.1 (C4′), 74.3 (C2′), 71.4 (C3′), 62.9 (C5′), 55.2 (MMTr), 37.8 (Lev), 37.7 (Lev), 29.8 (Lev), 29.7 (Lev), 27.6 (Lev), 27.5 (Lev).

2′,3′-Di-O-levulinoylribavirin (10). Compound 9 (7.17 mmol; 5.11 g) was treated with 80% aq AcOH (100 mL) overnight. The mixture was evaporated to dryness and the residue was purified by silica gel chromatography using gradient elution from 5 to 10% MeOH in DCM. Yield from compound II was 66%. ¹H NMR (CDCl₃+CD₃OD) δ 8.60 (s, 1H, H5), 7.34 (s, 1H, NH), 6.08 (d, J=4.3 Hz, 1H, H1′), 5.71 (dd, J=4.3 and 5.2 Hz, 1H, H2′), 5.58 (dd, J=4.3 and 5.2, 1H, H3′), 5.32 (s, 1H, NH), 4.35 (m, 1H H4′), 3.91 (dd, J=12.7 and 2.4 Hz, 1H, H5′), 3.77 (dd, J=12.7 and 2.8 Hz, 1H, H5″), 2.77-2.82 (m, 4H, Lev), 2.60-2.67 (m, 4H, Lev), 2.21 (s, 3H, Lev), 2.19 (s, 3H, Lev). ¹³C NMR (CDCl₃+CD₃OD) δ 207.0 (C═O Lev), 171.9 (C═O Lev), 171.4 (C═O Lev), 161.0 (C═O), 157.0 (C3), 144.7 (C5), 90.4 (C1′), 84.7 (C4′), 75.1 (C2′), 71.1 (C3′), 61.0 (C5′), 37.7 (Lev), 37.6 (Lev), 29.8 (Lev), 29.7 (Lev), 27.5 (Lev), 27.4 (Lev).

### RIBAVIRIN 5′-{O-[PHENYL-N—[(S)-2-METHOXY-1-METHYL-2-OXOETHYL]PHOSPHORAMIDATE (12)

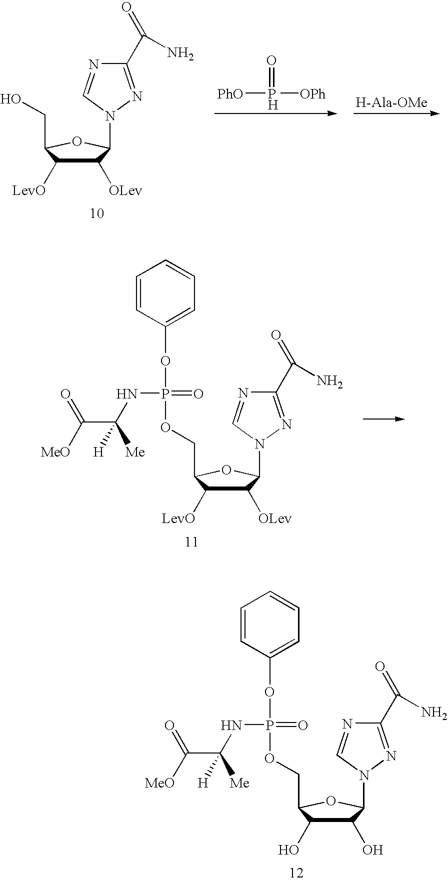

2′,3′-Di-O-levulinoylribavirin 5′-(O-[phenyl-N—[(S)-2-methoxy-1-methyl-2-oxoethyl]phosphoramidate (11). Compound 10 (0.41 mmol; 0.18 g) was coevaporated twice from dry pyridine, dissolved in the same solvent (3.0 mL) and diphenylphosphite (0.61 mmol; 118 μL) was added under nitrogen. After 20 min, L-alanine methyl ester (0.86 mmol; 0.12 g) dried by coevaporation from pyridine was added dissolved in a mixture of dry MeCN (4.0 mL) and pyridine (1.0 mL). Immediately after this addition, CCl₄ (2.5 mL) and distilled triethylamine (2.8 mmol; 400 μL) were added. The reaction was allowed to proceed for 70 min and the mixture was then evaporated to dryness. Silica gel chromatography by a gradient elution from 3 to 10% of MeOH in DCM gave compound II as a foam in 67% yield. ¹H NMR (CDCl₃) mixture of R_{P} ans S_{P} diastereomers δ 8.40 and 8.46 (2×s, 1H, H5), 7.34 and 7.37 (2×br s, 1H, NH₂), 7.10-7.30 (m, 5H, Ph), 6.38 and 6.46 (2×br s, 1H, NH₂), 6.09 and 6.10 (2×d, J=4.5 Hz, 1H, H1′), 5.71 and 5.73 (2×dd, J=4.5 and 5.0, 1H, H2′), 5.61 and 5.63 (2×dd, J=5.0 and 5.0, 1H, H3′), 4.45-4.49 (m, 1H, H4′), 4.28-4.43 (m, 2H, H5′ and H5″), 3.98-4.07 (m, 1H, H-Ala), 3.62 and 3.64 (2×s, 3H, MeO-Ala), 2.73-2.79 (m, 4H, Lev), 2.56-2.66 (m, 4H, Lev), 2.17 and 2.18 (2×s, 6H, Lev), 1.31 and 1.33 (2×d, J=7.2 Hz, 3H, Me Ala). ³¹P NMR (CDCl₃) 3.0 and 3.2.

Ribavirin 5′-{O-[phenyl-N—[(S)-2-methoxy-1-methyl-2-oxoethyl]phosphoramidate (12). Compound II (0.21 mmol; 0.14 g) was dissolved at 0° C. into a mixture of hydrazine hydrate (4.0 mmol; 124 μL), dry pyridine (4.0 mL) and AcOH (1.0 mL) and the reaction was allowed to proceed for 1 h. The unreacted hydrazine was quenched by acetone. The volatiles were removed under reduced pressure and the crude product was purified by silica gel chromatography increasing the MeOH content of DCM in a stepwise manner from 5% to 10% and then to 20%. Yield 60%. ¹H NMR (CD₃OD) mixture of R_{P} ans S_{P} diastereomers δ 8.72 and 8.74 (2×s, 1H, H5), 7.33-7.37 (m, 2H, Ph), 7.17-7.23 (m, 3H, Ph), 5.98 (2×d, J=3.5 Hz, 1H, H1′), 4.54 and 4.56 (2×dd, J=3.5 and 4.7 Hz, 1H, H2′), 4.47 (dd, J=4.7 and 5.9, 1H, H3′), 4.26-4.43 (m, 3H, H4′, H5′ and H5″), 3.91 and 3.94 (2×dd, J=9.3 and 7.2 Hz, H-Ala), 3.65 and 3.67 (2×s, 3H, MeO-Ala), 1.29 and 1.32 (2×d, J=7.2 Hz, 3H, Me Ala). ¹³C NMR (CD₃OD) δ 174.1 (C═O Ala), 161.9 (CONH₂), 157.1 (Ph), 150.7 (C3), 145.3 (C5), 92.4 (C1′), 83.1 (C4′), 74.8 (C2′), 70.2 (C3′), 65.9 (C5′), 51.9 (MeO-Ala), 49.8 (C-Ala), 19.1 (Me Ala). ³¹P NMR (CD₃OD) 3.8 and 4.0. HRMS: [M+H]⁺ obsd. 486.1389, calcd. 486.1384; [M+Na]⁺ obsd. 508.1206, calcd. 508.1204; [M+K]⁺ obsd. 524.0937, calcd. 524.0943.

### PROTECTED RIBAVIRIN (14)

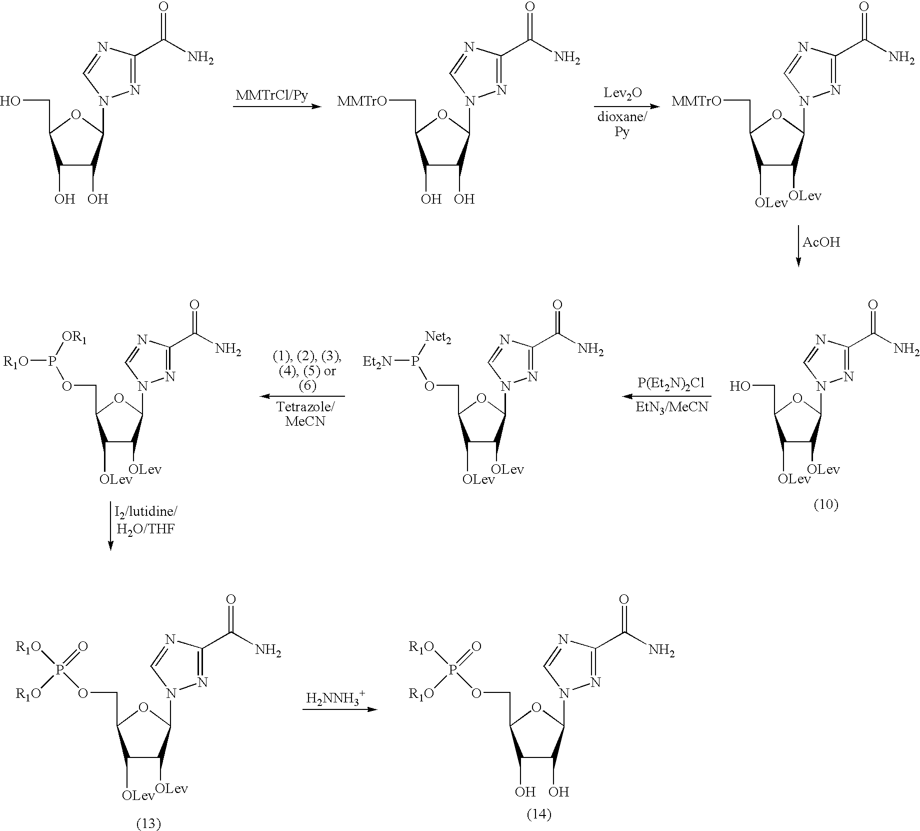

2′,3′-di-O-Levulinoylribavirin 5′-bis[3-acetyloxy-2,2-bis(ethoxycarbonyl)propyl]phosphate (13). 2′,3′-di-O-Levulinoylribavirin (3.1 mmol; 1.38 g), coevaporated from dry MeCN and stored on P₂O₅ for 24 h, was dissolved in dry DCM (6.0 mL) under nitrogen and bis(diethylamino)chlorophosphine (4.4 mmol; 0.92 mL) was added. After 2 hours, the reaction mixture was passed through a short silica gel column (dried in oven) eluting with ethyl acetate containing 0.5% triethylamine. The elute was evaporated to dryness and the residue was coevaporated three times from MeCN to remove the traces of triethylamine. The product was dissolved in dry MeCN (2.0 mL) and diethyl 2-acetyloxymethyl-2-hydroxymethylmalonate (4.3 mmol; 1.126 g) dried on P₂O₅ was added. The solution was mixed with a solution of tetrazole (7.8 mmol) in MeCN (17.3 mL). The reaction was allowed to proceed for 1 h. Iodine (1.61 mmol; 0.41 g) in a mixture of THF (6.0 mL), H₂O (3.0 mL) and 2,6-lutidine (1.5 mL) was added and the mixture was stirred overnight. Aqueous NaHSO₃ (50 mL of 5% solution) was added and the product was extracted in DCM (2×40 mL and 2×30 mL). The organic phase was dried on Na₂SO₄ and evaporated to dryness. The product was purified by silica gel chromatography using 10-15% MeOH in DCM as an eluent. The yield was 4%. ¹H NMR (CDCl₃) δ 8.42 (s, 1H, H5), 7.55 (s, 1H, NH), 6.06 (d, J=3.6 Hz, 1H, H1′), 6.01 (s, 1H, NH), 5.65 (dd, J=3.6 and 5.3 Hz, 1H, H2′), 5.50 (dd, J=5.3 and 5.5, 1H, H3′), 4.40-455 (m, 9H, 2×CH₂OP, 2×CH₂OAc and H4′), 4.15-4.25 (m, 10H, 4×OCH₂Me, H5′ and H5″), 3.77 (dd, J=12.7 and 2.8 Hz, 1H, H5″), 2.73-2.78 (m, 4H, Lev), 2.59-2.65 (m, 4H, Lev), 2.17 (s, 3H, Ac), 2.15 (s, 3H, Ac), 2.02 (s, 3H, Lev), 2.01 (s, 3H, Lev), 1.22 (q, J=7.0 Hz, 12H, 4×OCH₂CH₃). ¹³C NMR (CDCl₃) δ 206.3 (C═O Lev), 206.2 (C═O Lev), 171.6 (C═O Lev), 171.5 (C═O Lev), 171.3 (2×Ac), 170.2 (4×COOEt), 166.3 (C═O), 157.0 (C3), 144.7 (C5), 90.1 (C1′), 81.4 (C4′), 74.5 (C3′), 70.4 (C2′), 66.8 (C5′), 65.3 (CH₂OP), 65.2 (CH₂OP), 62.3 (4×OCH₂CH₃), 61.2 (CH₂OAc), 61.1 (CH₂OAc), 57.9 (2×spiro C), 37.6 (Lev), 29.7 (Lev), 27.4 (Lev), 20.6 (2×Ac), 13.9 (4×OCH₂CH₃).

Ribavirin 5′-bis[3-acetyloxy-2,2-bis(ethoxycarbonyl)propyl]phosphate (14). Compound 13 (0.10 mmol; 0.10 g) was treated with hydrazinium acetate (0.55 mL of 0.5 mol L⁻¹ in a 4:1 mixture of pyridine and AcOH) for 45 min. The reaction was quenched with acetone (20 μL). The crude product was purified by RP-HPLC (Hypersil ODS; 10×250 mm; 5 μm) using isocratic elution with 40% MeCN in H₂O. The yield was 73%. ¹H NMR (CDCl₃) δ 8.51 (s, 1H, H5), 7.62 (s, 1H, NH), 6.38 (s, 1H, NH), 6.00 (d, J=2.4 Hz, 1H, H1′), 5.05 (br s, 1H, OH), 4.43-4.62 (m, 11H, 2×CH₂OP, 2×CH₂OAc, H2′, H3′ and H4′), 4.18-4.30 (m, 11H, 4×OCH₂Me, OH, H5′ and H5″), 2.06 (s, 3H, Ac), 2.04 (s, 3H, Ac), 1.22-1.29 (m, 12H, 4×OCH₂CH₃). ¹³C NMR (CDCl₃) 170.4 (2×Ac), 166.3 (4×COOEt), 161.3 (C═O), 157.0 (C3), 144.9 (C5), 92.6 (C1′), 82.9 (C4′), 75.2 (C2′), 70.4 (C3′), 67.7 (C5′), 65.4 (2×CH₂OP), 62.4 (4×OCH₂CH₃), 61.3 (2×CH₂OAc), 57.9 (2×spiro C), 20.6 (2×Ac), 13.9 (4×OCH₂CH₃). MS [M+H]⁺ obsd. 813.6, calcd. 813.2; [M+Na]⁺ obsd. 835.5, calcd. 835.2; [M+K]⁺ obsd. 851.5, calcd. 851.2.

### PROTECTED 2′-O-METHYLCYTIDINE

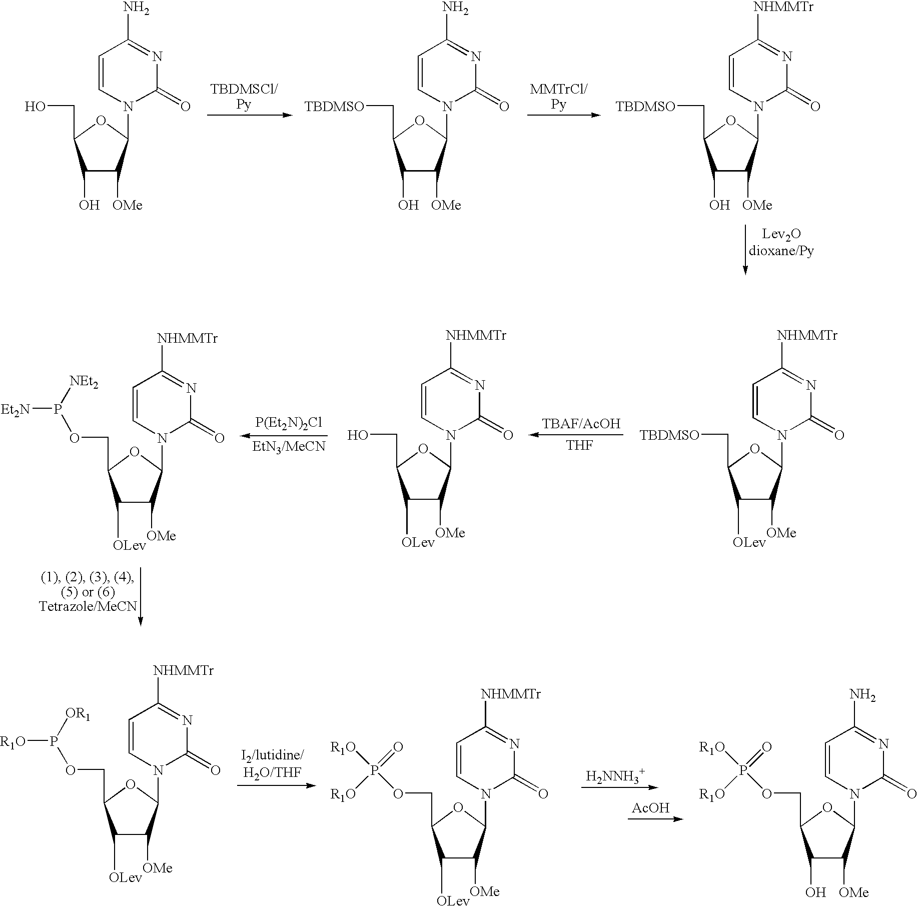

### DIETHYL 2-ACETYLOXYMETHYL-2-HYDROXYMETHYLMALONATE

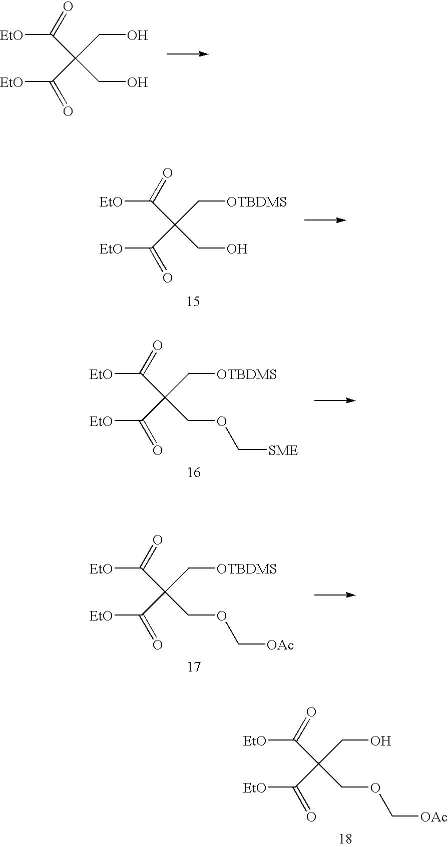

Diethyl 2-(tert-butyldimethylsilyloxymethyl)-2-hydroxymethylmalonate (15). Diethyl 2,2-bis(hydroxymethyl)malonate (28.3 mmol; 6.23 g) was coevaporated twice from dry pyridine and dissolved in the same solvent (20 mL). tert-Butyldimethylsilyl chloride (25.5 mmol; 3.85 g) in dry pyridine (10 mL) was added portionwise. The reaction was allowed to proceed for 4 days. The mixture was evaporated to a solid foam, which was then equilibrated between water (200 mL) and DCM (4×100 mL). The organic phase was dried on Na₂SO₄. The product was purified by silica gel chromatography eluting with 10% ethyl acetate in DCM. The yield was 78%. ¹H NMR (CDCl₃) δ 4.18-4.25 (m, 4H, OCH₂Me), 4.10 (s, 2H, CH₂OSi), 4.06 (s, 2H, CH₂OH), 2.63 (br s, 1H, OH), 1.26 (t, J=7.0 Hz, 6H, OCH₂CH₃), 0.85 (s, 9H, Si—SMe₃), 0.05 (s, 6H, Me-Si). ¹³C NMR (CDCl₃) δ 169.2 (C═O), 63.3 (CH₂OH), 62.8 (CH₂OSi), 61.6 (spiro C), 61.4 (OCH₂Me), 25.6 [C(CH₃)₃], 18.0 (Si—CMe₃), 14.0 (OCH₂CH₃), −3.6 (Si—CH₃). MS [M+H]⁺ obsd. 335.7, calcd. 335.2; [M+Na] obsd. 357.6, calcd. 357.2.

Diethyl 2-(tert-butyldimethylsilyloxymethyl)-2-methylthiomethylmalonate (16). Compound 15 (19.7 mmol; 6.59 g) was dissolved into a mixture of acetic anhydride (40 mL), acetic acid (12.5 mL) and DMSO (61 mL) and the mixture was stirred overnight. The reaction was stopped by dilution with cold aq Na₂CO₃ (290 ml 10% aq solution) and the product was extracted in diethyl ether (4×120 mL). The combined organic phase was dried on Na₂SO₄. The product was purified by silica gel chromatography using DCM as an eluent. The yield was 91%. ¹H NMR (CDCl₃) δ 4.61 (s, 2H, OCH₂S), 4.14-4.19 (m, 4H, OCH₂Me), 4.06 (s, 2H, CH₂OSi), 4.00 (s, 2H, CH₂OCH₂SMe), 2.06 (SCH₃), 1.22 (t, J=7.0 Hz, 6H, OCH₂CH₃), 0.83 (s, 9H, Si—SMe₃), 0.02 (s, 6H, Me-Si). ¹³C NMR (CDCl₃) δ 168.3 (C═O), 75.6 (CH₂S), 65.7 (CH₂OCH₂SMe), 61.4 (CH₂OSi), 61.2 (spiro C), 60.9 (OCH₂Me), 25.6 [C(CH₃)₃], 18.0 (Si—CMe₃), 14.0 (OCH₂CH₃), 13.7 (SCH₃), −3.6 (Si—CH₃). MS [M H]⁺ obsd. 395.4, calcd. 395.2; [M+Na]⁺ obsd. 417.6, calcd. 417.2.

Diethyl 2-acetyloxymethyl-2-(tert-butyldimethylsilyloxymethyl)malonate (17). Compound 16 (17.9 mmol; 7.08 g) was dissolved in dry DCM (96 mL) under nitrogen. Sulfurylchloride (21.5 mmol; 1.74 mL of 1.0 mol L⁻¹ solution in DCM) was added in three portions and the mixture was stirred for 70 min under nitrogen. The solvent was removed under reduced pressure and the residue was dissolved into dry DCM (53 mL). Potassium acetate (30.9 mmol; 3.03 g) and dibenzo-18-crown-6 (13.5 mmol; 4.85 g) in DCM (50 mL) were added and the mixture was stirred for one hour and a half. Ethyl acetate (140 mL) was added, the organic phase was washed with water (2×190 mL) and dried on Na₂SO₄. The product was purified by silica gel chromatography using DCM as an eluent. The yield was 71%. ¹H NMR (CDCl₃) δ 5.24 (s, 2H, OCH₂O), 4.15-4.22 (m, 4H, OCH₂Me), 4.13 (s, 2H, CH₂OSi), 4.08 (s, 2H, CH₂OAc), 2.08 (Ac), 1.26 (t, J=8.0 Hz, 6H, OCH₂CH₃), 0.85 (s, 9H, Si—SMe₃), 0.04 (s, 6H, Me-Si). ¹³C NMR (CDCl₃) δ 170.2 (Ac), 168.0 (C═O), 89.3 (OCH₂O), 67.5 (CH₂OAc), 61.4 (OCH₂Me), 61.1 (CH₂OSi), 60.2 (spiro C), 25.6 [C(CH₃)₃], 21.0 (Ac), 18.1 (Si—CMe₃), 14.0 (OCH₂CH₃), −5.7 (Si—CH₃). MS [M+Na]⁺ obsd. 429.6, calcd. 429.2.

Diethyl 2-acetyloxymethyl-2-hydroxymethylmalonate (18). Compound 17 (7.2 mmol; 2.93 g) was dissolved in dry THF (23 mL) and trietylamine trihydrogenfluoride (8.64 mmol; 1.42 mL) was added. The mixture was stirred for one week. Aq triethylammonium acetate (13 mL of 2.0 mol L⁻¹ solution) was added. The mixture was evaporated to dryness and the residue was purified by silica gel chromatography using DCM containing 2-5% MeOH as an eluent. The yield was 74%. ¹H NMR (CDCl₃) δ 5.25 (s, 2H, OCH₂O), 4.16-4.29 (m, 6H, OCH₂Me and CH₂OAc), 4.13 (s, 2H, CH₂OH), 2.10 (Ac), 1.81 (br s, 1H, OH), 1.26 (t, J=9.0 Hz, 6H, OCH₂CH₃). MS [M+Na]⁺ obsd. 315.3, calcd. 315.1.

### THYMIDINE 5′-BIS[3-ACETYLOXYMETHOXY-2,2-BIS(ETHOXYCARBONYL)PROPYL]PHOSPHATE

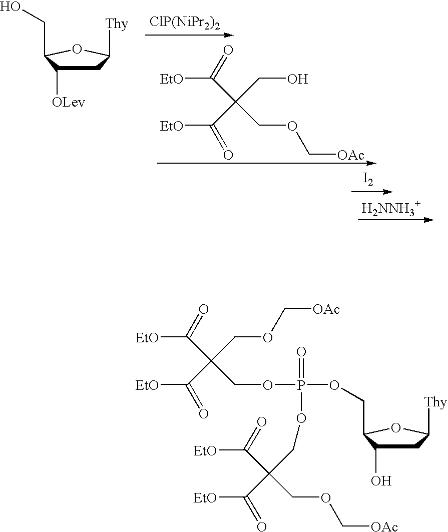

3′-O-Levulinoylthymidine (0.47 mmol; 0.166 g) was coevaporated once from dry pyridine and three times from dry MeCN and dissolved in dry DCM (1.2 mL) under nitrogen. Triethylamine (2.35 mmol; 0.34 mL) and bis(diethylamino)chlorophosphine (0.68 mmol; 0.145 mL) were added and the mixture was stirred under nitrogen for 2 h. The product was isolated by passing the mixture through a short silica gel column with a 4:1 mixture of ethyl acetate and hexane containing 0.5% triethylamine. The solvent was removed under reduced pressure and the residue was coevaporated three times from dry MeCN to remove the traces of triethylamine. The residue was dissolved in dry MeCN (1.0 mL) and 3-acetyloxymethoxy-2,2-bis(ethoxycarbonyl)propanol (1.68 mmol; 0.49 g) in dry MeCN (1.0 mL) and tetrazole (2.91 mmol; 6.46 mL of 0.45 mol L−1 solution in MeCN) were added under nitrogen. The reaction was allowed to proceed for 6 h and then iodine (0.73 mmol; 0.185 g) in a mixture of THF (4.0 mL), H₂O (2.0 mL) and 2,6-lutidine (1.0 mL) was added. The oxidation was allowed to proceed overnight. The excess of iodine was destroyed with 5% NaHSO₃. The mixture was extracted three times with DCM. The organic phase was washed with brine, dried on Na₂SO₄ and evaporated to dryness. The crude product was purified on a silica gel column eluting with DCM containing 5-10% MeOH. The yield was 15%.

3′-O-Levulinoylthymidine 5′-bis[3-acetyloxymethoxy-2,2-bis(ethoxycarbonyl)propyl]phosphate (0.071 mmol; 69 mg) was dissolved in dry DCM (2.0 mL) and hydrazine acetate (0.12 mmol; 11 mg) in dry MeOH (0.20 mL) was added. After 1 h, hydrazinium acetate (0.05 mmol; 4.6 mg) in a mixture of DCM (100 μL) and MeOH (20 μL) was added. The reaction was allowed to proceed for 2 h and the addition of hydrazinium acetate was repeated. The reaction was quenched with acetone and the mixture was evaporated to dryness. The product was purified on a silica gel column eluting first with ethyl acetate and then with DCM containing 15% MeOH. The yield was quantitative. ¹H NMR (CDCl₃) δ 8.91 (s, 1H, N3H), 7.34 (s, 1H, H6), 6.31 (dd, J=6.0 and 6.0 Hz, 1H, H1′), 5.25 (s, 4H, OCH₂O), 4.54 (m, 5H, 2×CH₂OCH₂OAc and H3′), 4.24 (m, 10H, 4×OCH₂Me, H5′ and H5′), 4.05 (s, 1H, H4′), 3.61 (br s, 1H, 3′-OH), 2.42 (m, 1H, H2′), 2.24 (m, 1H, H2′), 2.11 (s, 6H, 2×Ac), 1.95 (s, 3H, 5-Me), 1.27 (m, 12H, 4×OCH₂CH₃). ¹³C NMR (CDCl₃) δ 170.6 (Ac), 166.6 (COOEt), 163.7 (C4=O), 150.3 (C2=O), 135.5 (C6), 111.4 (C5), 88.8 (OCH₂O), 84.8 (C4′), 84.4 (C1′), 70.7 (C3′), 67.2 (POCH₂), 67.0 (C5′), 65.3 (CH₂OCH₂Oac), 62.3 (OCH₂Me), 58.8 (spiro C), 39.6 (C2′), 20.9 (Ac), 13.9 (OCH₂CH₃), 12.4 (5-Me). ³¹P NMR (acetone) δ −2.1 ppm. MS [M+Na]⁺ obsd. 893.8, calcd. 893.3.

### Antiviral Activity of Selected Compounds

### HCV Replicon Assay

Antiviral activity of the test compounds was assessed (Okuse, et al., Antivir. Res. (2005) 65:23) in the stably HCV RNA-replicating cell line, AVA5 (genotype 1b, subgenomic replicon, Blight, et al., Sci. (2000) 290:1972). Compounds were added to dividing cultures daily for three days. Cultures generally start the assay at 30-50% confluence and reach confluence during the last day of treatment. Intracellular HCV RNA levels and cytotoxicity were assessed 72 hours after treatment.

Quadruplicate cultures for HCV RNA levels and cytoxicity (on 96-well plates) were used. A total of 12 untreated control cultures, and triplicate cultures treated with α-interferon (concentrations of: 10 IU/mL, 3.3 IU/mL, 1.1 IU/mL and 0.37 IU/mL) and 2′C-Me-C (concentrations of: 30 μM, 10 μM, 3.3 μM and 1.1 μM) served as assay controls.

Intracellular HCV RNA levels were measured using a conventional blot hybridization method, in which HCV RNA levels are normalized to the levels of β-actin RNA in each individual culture (Okuse, et al., Antivir. Res. (2005) 65:23). Cytotoxicity was measured using an established neutral red dye uptake assay (Korba and Gerin, Antivir. Res. (1992) 19:55; Okuse, et al., Antivir. Res. (2005) 65:23). HCV RNA levels in the treated cultures are expressed as a percentage of the mean levels of RNA detected in untreated cultures. The absorbance of the internalized dye at 510 nM (A₅₁₀) was used for quantitative analysis.

Compounds were dissolved in 100% tissue culture grade DMSO (Sigma, Inc.) at 10 mM. Aliquots of test compounds sufficient for one daily treatment were made in individual tubes and all material was stored at −20° C. For the test, the compounds were suspended into culture medium at room temperature, and immediately added to the cell cultures. Compounds were analyzed separately in two groups with separate assay controls. The concentrations of the test compounds were run at concentrations of 10 μM, 3.3 μM, 1.1 μM and 0.37 μM.

Values presented (±standard deviations [S.D.]) were calculated by linear regression analysis using data combined from all treated cultures. S.D. was calculated using the standard error of regression generated from the linear regression analyses (QuattroPro™). EC₅₀ and EC₉₀, drug concentrations at which a 2-fold, or a 10-fold depression of HCV RNA (relative to the average levels in untreated cultures), respectively, were observed; CC₅₀, drug concentrations at which a 2-fold depression of neutral red dye uptake (relative to the average levels in untreated cultures) were observed.

As shown by the results in Table 1, compound 12 was inactive. By comparison, compound 14 was markedly more active compared to compound 12. These results demonstrate the ability of the 2,2-disubstituted-acyl(oxyalkyl) groups to neutralize the charge on the phosphate for entry into the cell but also their ability to be removed once inside the cell.

TABLE 1

| Compound | CC₅₀ (μM) | EC₅₀ (μM) | EC₉₀ (μM) |
|---|---|---|---|
| 14 | 9.7 ± 0.2 | 1.6 ± 0.2 | 4.4 ± 0.4 |
| 12 | >100 | >100 | >100 |

### Kinetic Studies

Preparation of the cell extract. 10×10⁶ of human prostate carcinoma cells (PC3) are treated with 10 mL of RIPA-buffer [15 mM Tris-HCl pH 7.5, 120 mM NaCl, 25 mM KCl, 2 mM EDTA, 2 mM EGTA, 0.1% Deoxycholic acid, 0.5% Triton X-100, 0.5% PMSF supplemented with Complete Protease Inhibitor Cocktail (Roche Diagnostics GmBH, Germany)] at 0° C. for 10 min. Most of the cells are disrupted by this hypotonic treatment and the remaining ones are disrupted mechanically. The cell extract obtained is centrifuged (900 rpm, 10 min) and the pellet is discarded. The extract is stored at −20° C.

Stability of protected nucleotide analogs in the cell extract. The cell extract is prepared as described above (1 mL), and is diluted with a 9-fold volume of HEPES buffer (0.02 mol L⁻¹, pH 7.5, 10.1 mol L⁻¹ with NaCl). A protected nucleotide analog (0.1 mg) is added into 3 mL of this HEPES buffered cell extract and the mixture is kept at 22±1° C. Aliquots of 150 μL are withdrawn at appropriate intervals, filtered with SPARTAN 13A (0.2 μm) and cooled in an ice bath. The aliquots are analyzed immediately by HPLC-ESI mass spectroscopy (Hypersil RP 18, 4.6×20 cm, 5 μm). For the first 10 min, 0.1% aq formic acid containing 4% MeCN is used for elution and then the MeCN content is increased to 50% by a linear gradient during 40 min.

Stability of protected nucleotide analogs towards Porcine Liver Esterase. A protected nucleotide analog (1 mg) and 3 mg (48 units) of Sigma Porcine Liver Esterase (66H7075) are dissolved in 3 mL of HEPES buffer (0.02 mol L⁻¹, pH 7.5, I=0.1 mol L⁻¹ with NaCl). The stability test is carried out as described above for the cell extract.

Stability tests in human serum. Stability tests in human serum are carried out as described for the whole cell extract. The measurements are carried out in serum diluted 1:1 with HEPES buffer (0.02 mol L⁻¹, pH 7.5, I=0.1 mol L⁻¹ with NaCl).

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

What is claimed is:

1. A compound of Formula (I) or a pharmaceutically acceptable salt, prodrug or prodrug ester: 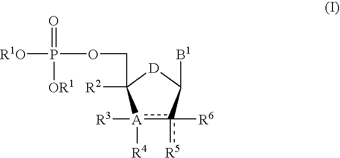
wherein: [symbol]
is a double or single bond;
A is selected from the group consisting of C, O and S;
B¹ is an optionally substituted heterocyclic base or a derivative thereof;
D is C═CH₂ or O;
each R¹ is independently absent, hydrogen or 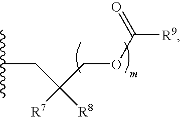
provided that at least one R¹ is 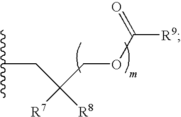
R² is selected from the group consisting of hydrogen, azido, —CN, an optionally substituted C₁₋₄ alkyl and an optionally substituted C₁₋₄ alkoxy;
R³ is absent or selected from the group consisting of hydrogen, halogen, hydroxy and an optionally substituted C₁₋₄ alkyl;
R⁴ is absent or selected from the group consisting of hydrogen, halogen, azido, amino and hydroxy;
R⁵ is selected from the group consisting of hydrogen, halogen, hydroxy, —CN, —NC, an optionally substituted C₁₋₄ alkyl and an optionally substituted C₁₋₄ alkoxy;
R⁶ is absent or selected from the group consisting of hydrogen, halogen, hydroxy, —CN, —NC, an optionally substituted C₁₋₄ alkyl, an optionally substituted haloalkyl and an optionally substituted hydroxyalkyl, or when the bond to R⁵ indicated by [symbol] is a double bond, then R⁵ a C₁₋₄ alkenyl and R⁶ is absent;
R⁷ and R⁸ are each independently —C≡N or an optionally substituted substituent selected from the group consisting of C₁₋₈ organylcarbonyl, C₁₋₈ alkoxycarbonyl and C₁₋₈ organylaminocarbonyl;
R⁹ is hydrogen or an optionally substituted C₁₋₄-alkyl; and
m is 1 or 2.

2. The compound of claim 1, wherein R⁷ is —C≡N and R⁸ is an optionally substituted C₁₋₆ alkoxycarbonyl.

3. The compound of claim 1, wherein R⁷ is —C≡N and R⁸ is an optionally substituted C₁₋₆ organylaminocarbonyl.

4. The compound of claim 1, wherein both R⁷ and R⁸ are an optionally substituted C₁₋₆ organylcarbonyl.

5. The compound of claim 1, wherein both R⁷ and R⁸ are an optionally substituted C₁₋₆ alkoxycarbonyl.

6. The compound of claim 1, wherein R⁹ is an optionally substituted C₁₋₄-alkyl.

7. The compound of claim 1, wherein 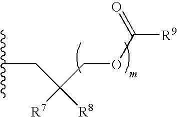
is selected from the group consisting of:

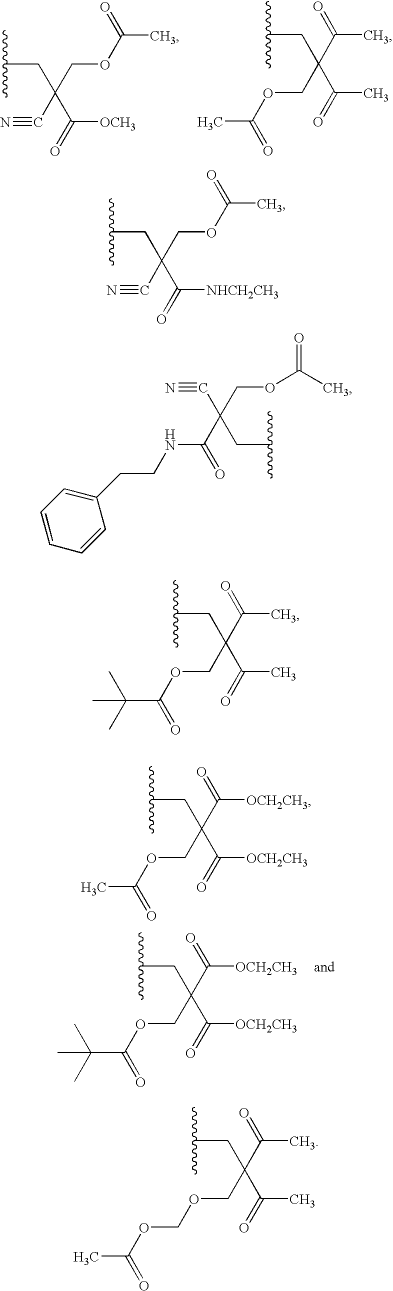

8. The compound of claim 7, wherein

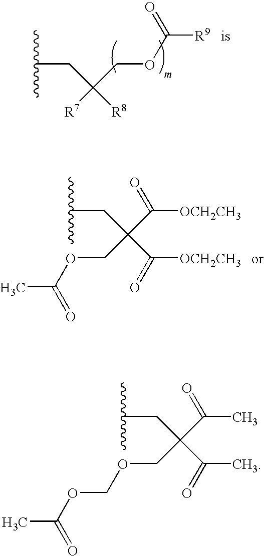

9. The compound of claim 1, wherein both R¹ groups are the same.

10. The compound of claim 1, wherein B¹ is selected from the group consisting of: 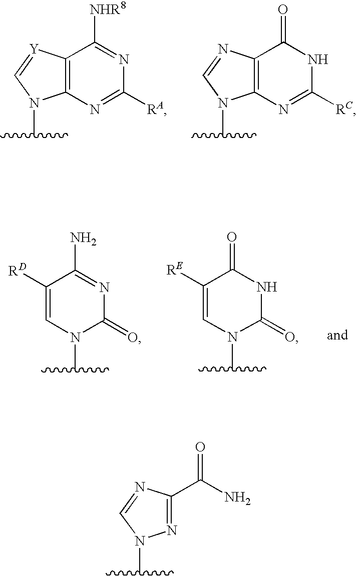
wherein:
R^{A} is hydrogen or halogen;
R^{B} is hydrogen, an optionally substituted C₁₋₄ alkyl, or an optionally substituted C₃₋₈ cycloalkyl;
R^{C} is hydrogen or amino;
R^{D} is hydrogen or halogen;
R^{E} is hydrogen or an optionally substituted C₁₋₄alkyl; and
Y is N or CR^{F}, wherein R^{F} hydrogen, halogen or an optionally substituted C₁₋₄-alkyl.

11. The compound of claim 10, wherein B¹ is selected from the group consisting of:

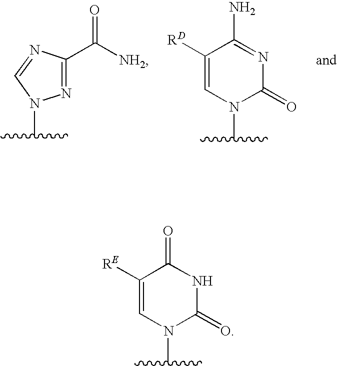

12. The compound of claim 1, wherein the compound of Formula (I) is selected from the group consisting of:

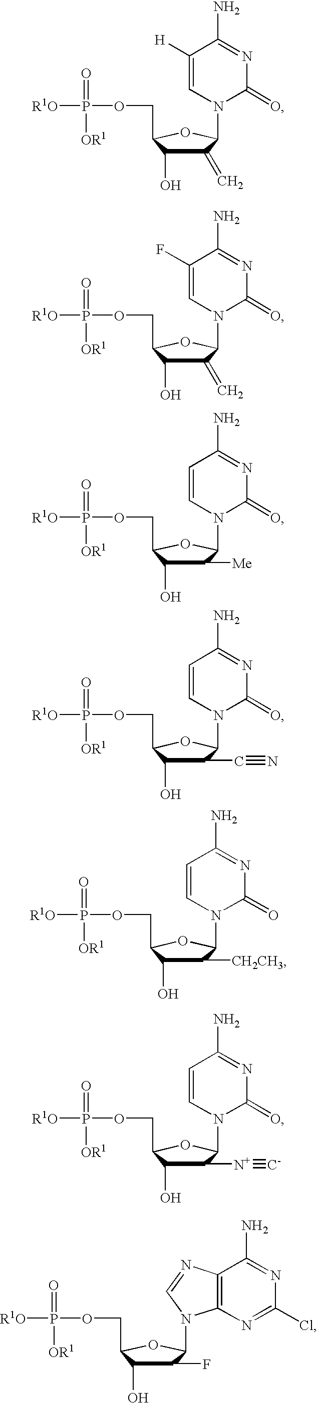

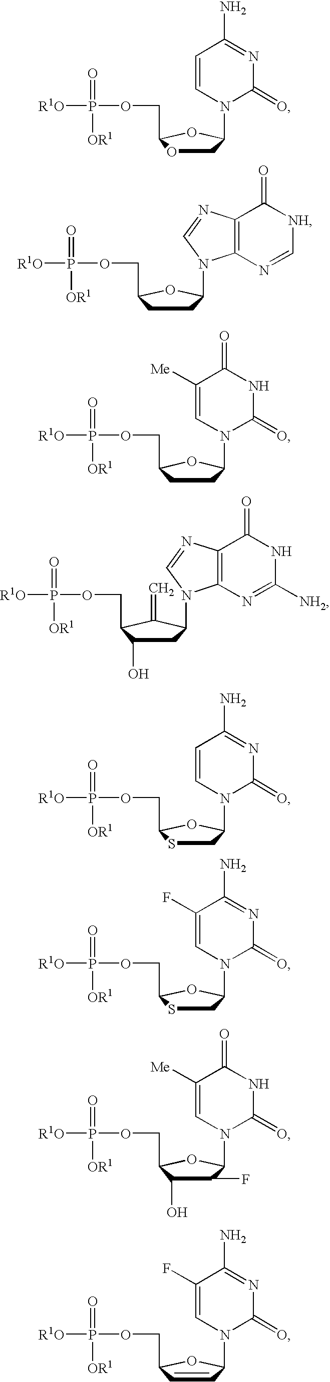

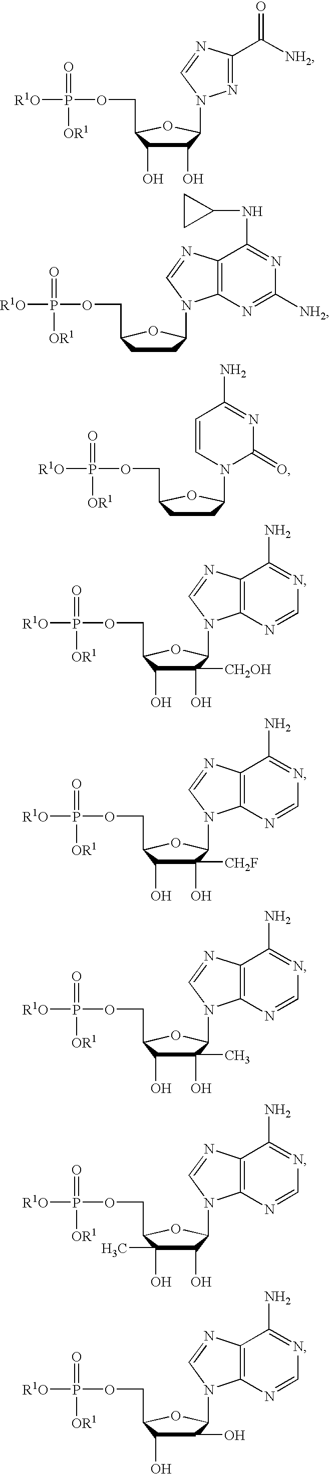

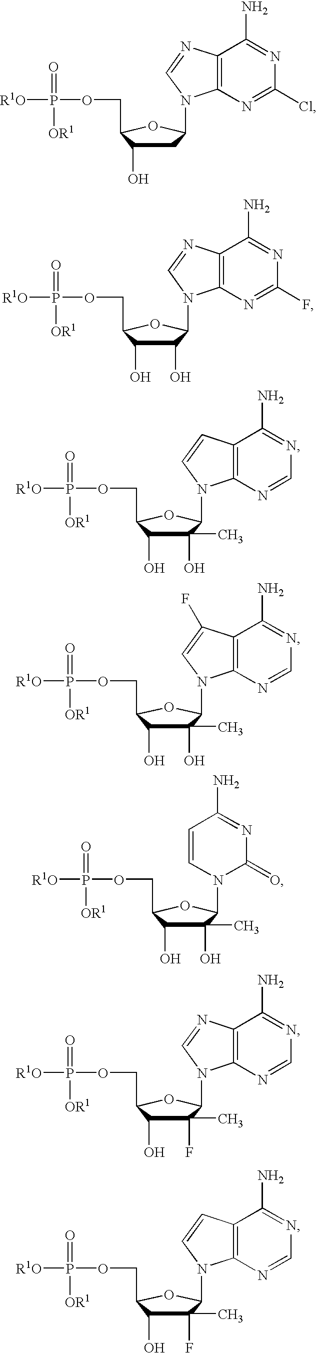

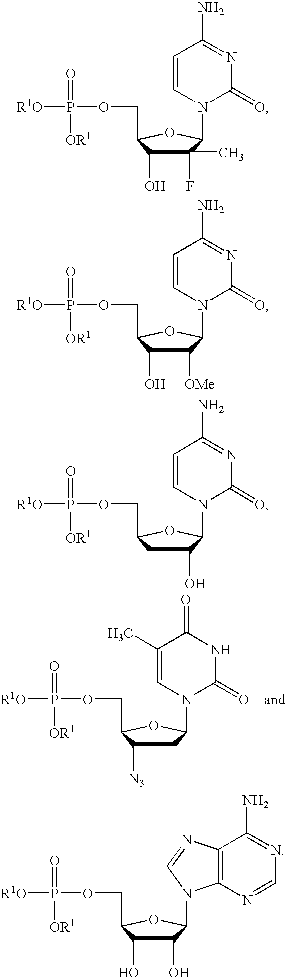

13. The compound of claim 1, wherein the compound of Formula (I) is:

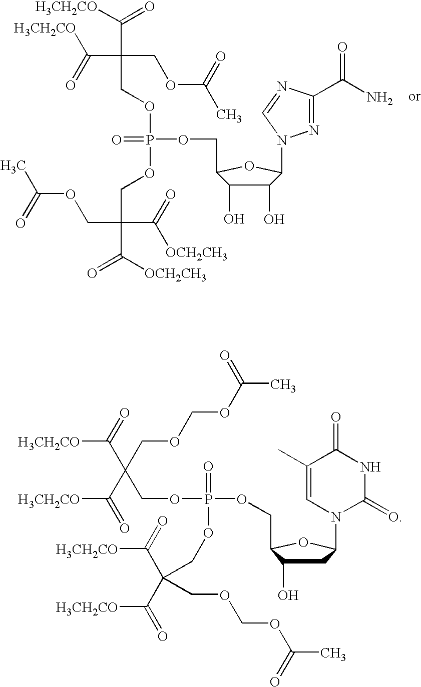

14. A compound of Formula (II) or a pharmaceutically acceptable salt, prodrug or prodrug ester: 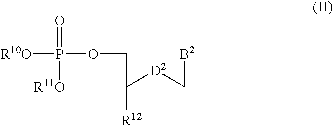
wherein:
B² is an optionally substituted heterocyclic base or a derivative thereof;
D² is O or —CH₂—;
each R¹⁰ and R¹¹ are each independently absent, hydrogen or 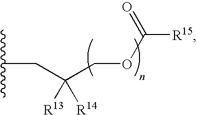
provided that at least one of R¹⁰ and R¹¹ are 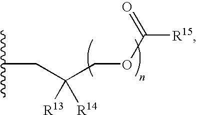
R¹² is hydrogen or —(CH₂)—OH;
R¹³ and R¹⁴ are each independently —C≡N or an optionally substituted substituent selected from the group consisting of C₁₋₈ organylcarbonyl, C₁₋₈ alkoxycarbonyl and C₁₋₈ organylaminocarbonyl;
R¹⁵ is hydrogen or an optionally substituted C₁₋₄-alkyl; and
n is 1 or 2.

15. A pharmaceutical composition comprising a compound of claim 1, and a pharmaceutically acceptable carrier, diluent, excipient or combination thereof.

16. A method of ameliorating or treating a viral infection comprising administering to a subject suffering from the viral infection a therapeutically effective amount of a compound of claim 1.

17. A method comprising reacting a first reactant, wherein the first reactant comprises a nucleoside with a phosphoamidite attached to the 5′-carbon or a protected nucleoside derivative with a phosphoamidite attached to the 5′-carbon, with a second reactant that comprises a compound of Formula (G) having the structure 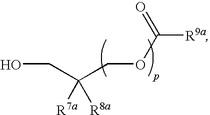
to form a compound having the structure 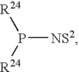
wherein at least one R²⁴ has the formula 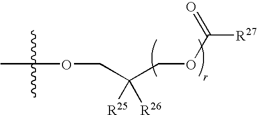
wherein R^{7a}, R^{8a}, R²⁵ and R²⁶ are each independently —C≡N or an optionally substituted substituent selected from the group consisting of C₁₋₈ organylcarbonyl, C₁₋₈ alkoxycarbonyl and C₁₋₈ organylaminocarbonyl; R^{9a} and R²⁷ are each independently hydrogen or an optionally substituted C₁₋₄-alkyl; p is 1 or 2; r is 1 or 2; NS² is a nucleoside or a protected nucleoside derivative; and the other R²⁴ is a biolabile group.

18. A method comprising, oxidizing the phosphorus in a compound having the structure 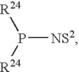
wherein at least one R²⁴ has the formula 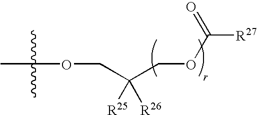
wherein R²⁵ and R²⁶ are each independently —C≡N or an optionally substituted substituent selected from the group consisting of C₁₋₈ organylcarbonyl, C₁₋₈ alkoxycarbonyl and C₁₋₈ organylaminocarbonyl; R²⁷ is hydrogen or an optionally substituted C₁₋₄-alkyl; r is 1 or 2; NS² is a nucleoside or a protected nucleoside derivative; and the other R²⁴ is a biolabile group, to phosphorus (V).

19. A method comprising reacting a first reactant, wherein the first reactant comprises a phosphite, with a second reactant that comprises a compound of Formula (G) having the structure 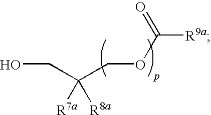
and a third reactant comprising a nucleoside or a protected nucleoside derivative; to form a compound having the structure 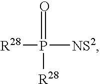
wherein at least one R²⁸ has the formula 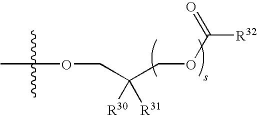
wherein R^{7a}, R^{8a}, R³⁰ and R³¹ are each independently —C≡N or an optionally substituted substituent selected from the group consisting of C₁₋₈ organylcarbonyl, C₁₋₈ alkoxycarbonyl and C₁₋₈ organylaminocarbonyl; R^{9a} and R³² are each independently hydrogen or an optionally substituted C₁₋₄-alkyl; p is 1 or 2; s is 1 or 2; NS² is a nucleoside or a protected nucleoside derivative; and the other R²⁸ is a biolabile group.

20. A compound selected from the group consisting of: [chemical structure]
